(19) United States
(12) Patent Application Publication
(10) Pub. No.: US 2020/0054021 A1
(43) Pub. Date: Feb. 20, 2020

(54) A PROCESS OF CONSTRUCTING SPECIFIC FUNCTIONAL MICROBIOMES FOR PROMOTING PLANT GROWTH, PLANT AND SOIL HEALTH, BIOCONTROL AND BIOREMEDIATION

(71) Applicant: MICROGEN BIOTECH, Carlow (IE)

(72) Inventors: Xuemei GERMAINE, Graiguecullen, Carlow (IE); Mary Jo HURLEY, Graiguecullen, Carlow (IE); Kieran GERMAINE, Graiguecullen, Carlow (IE)

(21) Appl. No.: 16/487,807

(22) PCT Filed: Feb. 22, 2017
(86) PCT No.: PCT/EP2017/054074

§ 371 Date: Aug. 21, 2019

(51) Int. Cl. A01N 63/00; C12N 1/20; C12Q 1/689

(52) U.S. Cl. CPC A01N 63/00; C12Q 2600/158; C12Q 1/689; C12N 1/20

(57) ABSTRACT

The present invention relates to the process of constructing plant-site specific functional microbiomes to screen, identify plant and soil microbes with beneficial traits, and to apply the functional microbiome, microbe or microbial consortia for improving the growth and health of plants cultivated on normal, stressed or marginal lands and for improving soil health, the removal and/or stabilisation of organic and inorganic pollutants, and the enhancement of soil microbial ecosystem functions.

### FIELD OF INVENTION

The present invention relates to a process of constructing functional microbiomes and to the isolation of plant and soil microbes with beneficial traits for improving the growth and health of plants and for improving soil health, the removal and/or stabilisation of organic and inorganic pollutants, and the enhancement of soil microbial ecosystem functions. The plants may include cereal, vegetable, fibre, fruit, ornamental, floral, scent, turf, bioenergy, biopharmaceutical and phytoremediation plants which may be cultivated on normal, stressed or marginal lands.

### BACKGROUND

It has been projected by the UN Food and Agricultural Organisation (FAO) that by 2050 the world will need to produce 70% more food in order to sustain its population. This unprecedented demand for food will require an additional land use of 2.7-4.9 MHa per year. This will also require innovations in agriculture to increase safe crop production from the current productive agricultural land, and from stress and pollution impacted arable land.

65% of agricultural land has deteriorated, been salinized, or impoverished in the past 50 years due to intensive agricultural production and the use of chemical fertilizers. Large areas of agricultural soil contaminated by organic and inorganic pollutants have been reported globally, especially in developing countries due to rapid industrial development and lack of sufficient environmental protection. In China, it's estimated that 20 million hectares of arable land (one fifth of the total agricultural land) has been impacted by heavy metals as a result of uncontrolled mining activity and irrigation with industrial wastewater. This has resulted in a reduction in food production of more than 10 million tons /year and in 12 million tons of food being contaminated with heavy metals in China annually. This can feed 40 million people, and results in a direct economic loss of more than $3 billion per year. Toxic amounts of heavy metals can enter the food chain through contaminated soils and accumulate in humans which can pose severe health risk.

Maximising productivity and protecting the food safety of crops produced on stressed and polluted agricultural land has become a critical target for sustainably feeding 900 million people by 2050. While crop yield had increased in the past decades via of the application of chemical fertilisers, pesticides and technologies such as the genetic modification (GM) of crops, the environmental and social impacts of the application of such chemicals and GM technologies has caused considerable public concern.

In the last decade, the importance and impact that microbes exert on human, plant and environmental health had been recognized. The advent of low-cost genome and microbiome sequencing technology, proteomics and metabolomics has exponentially increased the quality and quantity of genetic and functional information regarding the diversity and roles that microbes play.

In the past, the application of innovative naturally derived plant growth promoting (PGP) microbes had been shown to promote crop yield and soil fertility, protect crops from disease, increase food nutritional quality and improve food safety. The mechanisms of PGP are varied and many PGP microbes can produce a number of beneficial traits including plant hormones such as auxin and gibberellins, promote the acquisition of key nutrients, for example, nitrogen in the form of ammonium through nitrogen fixing PGP traits and phosphorous through the solubilisation of both organic and mineral phosphates. The ability to alleviate the stress response is an important trait of some PGP microbes that possess the enzyme Amino-Cyclopropane-Carboxylic acid (ACC) deaminase as this reduces ethylene levels produced as a result of stress, and also can affect other pathways within the plant which can improve the plants resistance to herbivore pests and improve tolerance to salt and drought stress as well as heavy metal contaminants. Some PGP have traits to degrade organic pollutants and stabilize inorganic pollution to promote plant growth and protect food safety. However, despite the potential of PGP microbes, commercial success is limited to a relatively small range of species with limited PGP traits and inconsistent efficacy when applied in different plant species, climate and environmental settings. In the last 5 years, major Agri-biotech companies have recognised the need for innovation to maximize the revolutionary benefits that PGP microbes for model agriculture. Precision agriculture is no longer a futuristic idea. To maximise the plant yield potential of PGP microbes, the microbe or microbial consortia need to be adapted precisely to a specific plant growing under specific site conditions. This will require a highly efficient process to enrich, isolate and identify microbes with the desired specific traits. However, the conventional methods for identifying microbes with multiple beneficial traits and developing microbial products normally takes 3-5 years with associated high costs.

This invention discloses a fast efficient process, Constructed Functional Microbiome (CFM) to identify large pools of microbes with multiple desired beneficial traits for specific plants and sites. The identified microbe or microbial consortia can be immediately developed as a commercial product for promoting the growth of targeted plants at specific sites which will lead to quick access to market. These identified microbes can be further developed for generic application for specific crops or for specific site conditions.

As used herein the terms constructed functional microbiome or microbial consortium means a microbial community of individual microbial species or strains of species that carry out a common function or which are involved with or lead to a plant phenotypic trait or other measurable plant parameter. There may be a symbiotic relationship between the organisms in the microbiome or microbial consortium.

### OBJECT OF INVENTION

Thus the object of this invention is to provide a platform process of Constructing Functional Microbiomes (CFM) to identify microbes with beneficial traits for use in promoting plant growth, plant and soil health, biocontrol and bioremediation. A further object is to utilize these constructed functional microbiomes and isolated microbes with beneficial traits on plants and soils. It is also an object to provide a site-specific, fast and productive process to isolate microbes and produce compositions comprising one or more desirable microbes having beneficial traits that can be applied in agriculture and bioremediation. In agriculture it is desirable that the constructed microbiome, the isolated microbes and the compositions can promote plant growth, improve and protect plant health under stressed conditions, improve soil health, and/or degrade/immobilise organic and inorganic pollutants. In bioremediation, it is a still further object that the constructed microbiome, the isolated microbes and the compositions can degrade organic pollution, or immobilise/solubilise heavy metals. A still further object is that the process provides for the production of functional microbiome compositions, isolated microbes and compositions which are site specific (i.e. bespoke, tailored). A further object is to provide a fast, high throughput, effective process to produce site specific functional microbiomes or microbial compositions in 2 to 4 months which is significantly more rapid than conventional methods. A further object is to produce generic microbe or microbial consortia with desired traits for a targeted plant or for two or more plant species on a specific site condition.

### SUMMARY OF INVENTION

According to the present invention, there is provided a method of constructing a functional microbiome, the microbiome comprising microbes with one or more beneficial traits comprising:

(a) collecting one or more of plant, rhizosphere or bulk soil samples from one or more agricultural or potential agricultural sites; the plant sample comprisinga samples of at least one of the root, rhizome, shoot, flower, seed, seedling, fruit, stem, cuttings or leaves,

(b) liberating any microorganisms present in the sample into a liquid medium,

(c) culturing any microorganisms present into an enrichment liquid medium to identify functional microbiomes with one or more specific beneficial traits,

(d) plating out the functional microbiome on a solid selection medium with a trait and selecting isolate for testing.

The functional microbiome of step (c) may go directly to the next step, or may go through a series of sequential or parallel enrichments with each enrichment step selecting for the same or a further additional trait to construct functional microbiomes with one or multiple traits.

With or without purifying the isolate, the isolate may be tested for additional beneficial traits on solid selective medium. If the isolate is purified, one or more microbes with one or more specific beneficial traits for application may be selected. If the isolate is not purified, the isolates with desired traits may be selected.

Suitably the process further involves purifying the isolates and testing the purified microbes for beneficial traits on solid selective medium. Preferably one or more purified microbes with one or more specific beneficial traits for application is then selected.

Preferably the construction of the functional microbiome is site specific.

Suitably the beneficial traits of the microbes include the promotion of plant growth and health, food safety and bioremediation.

Plant samples collected may be growing on non-stressed or stressed soil (soils from agricultural and non-agricultural regions subjected to drought, high salinity, and/or organic and/or inorganic pollutants). The selected plants may include, but are not limited to, human or animal crop plants (e.g. cereals or vegetables or fruits), plants used in agriculture (e.g. grasses, legumes fibre crops), biofuel crops, weeds, trees or shrubs growing on these sites. Bulk soil samples can be taken from the same sites. Preferably the plant and soil samples are collected from the area in which the functional microbiome, the microbe or microbial consortia is ultimately to be used.

Preferably at least two of the root, rhizome, shoot, flower, seed, seedling, fruit, stem, cuttings or leaves of the plant or the soil attached to the plant, or the bulk soil from an agricultural site or potential agricultural site or non cultural site are sampled. Preferably at least three of the root, rhizome, shoot, flower, seed, seedling, fruit, stem, cuttings or leaves of the plant or the soil attached to the plant, or the bulk soil from an agricultural site or potential agricultural site or non cultural site are sampled. Preferably at least four of the root, rhizome, shoot, flower, seed, seedling, fruit, stem, cuttings or leaves of the plant or the soil attached to the plant, or the bulk soil from an agricultural site or potential agricultural site or non cultural site are sampled. Preferably at least five of the root, rhizome, shoot, flower, seed, seedling, fruit, stem, cuttings or leaves of the plant or the soil attached to the plant, or the bulk soil from an agricultural site or potential agricultural site are sampled. Preferably all of the root, rhizome, shoot, flower, seed, seedling, fruit, stem, cuttings or leaves of the plant or the soil attached to the plant, or the bulk soil from an agricultural site or potential agricultural site or non cultural site are sampled. Suitable samples are taken from the rhizosphere soil, rhizoplane, phyllosphere and endosphere. The soil from the rhizosphere, or the bulk soil from the target site may be sampled.

The colonies of microorganisms which grow on the selective medium (one trait) are purified or not purified. Preferably, the colonies are not subjected to purification or isolation, but are directly tested to determine if they have one or more additional beneficial traits. Whilst not wishing to be bound by any theory, the inventors believe that colonies growing on the solid selective medium may comprise more than one organism which may exist in a cooperative or synergistic state which enhances their ability to perform the beneficial trait. Thus separating organisms which exist co-operatively is not desirable at this initial selection process. Also, 10 times greater numbers of the colonies can be selected and screened in a high throughput manner. After identifying the unpurified microbes with one or more multiple beneficial traits, these microbes are subject to further purification and the purified microbes are tested to determine if they have one or more beneficial traits. The process for selection first of the non-purified colonies enhances the possibility of making the most compatible microbial consortia (since the organisms prefer to co-exist in the non-purified state) and then using the purified microbes for field application. This will enhance the subsequent selection of the composition of the microbial consortia.

The term microorganism or microbe as used herein is defined broadly and includes bacteria and archaea as well as eukaryotic fungi and protists.

Suitably plant and soil samples are sampled and are transported back to the laboratory.

The plant material may be transferred to a sterile blender, sterile buffered diluent containing detergent added and the sample homogenised. Preferably the homogenised sample is shaken at high speed to liberate the bacteria.

The homogenised sample may be subjected to low speed centrifugation to remove the solid plant tissue. The clarified supernatant may be collected and subjected to high speed centrifugation to pellet the liberated microbial cells. Preferably the supernatant is removed and the pelleted microbiome is re-suspended in buffer.

The microbial cells may then be washed in the same buffered diluent, centrifuged, and re-suspended in buffered diluent.

Aliquots of the extracted microbiome may be stored for future use, and can be used for total DNA extraction for 16S rDNA and metagenomic profiling.

The subset of microbes within the extracted microbiome that possess specific traits are preferably selectively enriched in liquid cultures.

The desirable traits include but are not limited to the following;

Inorganic and organic phosphate and potassium release;

Diazotrophic (nitrogen fixing) activity;

Plant hormone production (indole-3-acetic acid, cytokinins, giberillins);

Plant stress hormone reduction (reduction in levels of ethylene through the degradation of 1-amino-1-cyclopropane carboxylate (ACC) due to the action of the bacterial enzyme ACC deaminase and the reduction in the level of abscisic acid in the plant roots,

The ability to degrade toxic organic compounds in the soil including pesticides (insecticides, herbicides and fungicides), mineral oils, polycyclic aromatic hydrocarbons (PAHs), nitro aromatic compounds, halogenated and non-halogenated aromatics and aliphatic compounds, The ability to sequester, accumulate, solubilise or immobilise toxic heavy metals (including lLead (Pb), Cadmium (Cd), Arsenic (As), Selenium (Se), Chromium (Cr), Zinc (Zn), Copper (Cu), Nickel (Ni), Cobalt (Co) and Mercury (Hg),

The ability to survive and grow in high saline conditions.

One embodiment of the invention for plant growth promotion of a garget plant on a specific stressed or non stressed soil condition is shown in FIG. 1.

Constructing a Phosphate Solubilising Functional Microbiome:

A sample of the extracted microbiome is inoculated into a flask containing sterile National Botanical Research Institute Phosphate (NBRIP) broth supplemented with tricalcium phosphate. The flask is shaken at high speed (200 rpm), at 10 to 30° C. for 3 to 10 days, suitably 7 days. The liquid culture may then be centrifuged to collect the selected microbiome, washed in buffer and re-suspended in 1/5 volume buffer. One suitable buffer is Ringer's buffer.

A portion of this suspension may be inoculated into a fresh flask of National Botanical Research Institute Phosphate (NBRIP) broth supplemented with tricalcium phosphate. The flask may again be shaken at high speed (200 rpm), at 10-30° C. suitably 20° C. for 3 to 10 days, suitably 7 days; Samples of this enrichment may be plated onto NBRIP agar containing tricalcium phosphate and bromophenol blue indicator. These agar plates are incubated at 10 to 30° C., suitably 20° C. for 3 to 10 days, suitably for 5 to 7 days.

The enriched functional microbiome is stored at −80° C. Large isolated colonies from the enriched microbiome showing significant discolouration are collected and stored at −80° C.

Constructing a IAA (Iodoacetanilide) Producing Functional Microbiome:

A sample of the extracted microbiome may be inoculated into a flask containing sterile nitrogen free Dworkin and Foster minimal media broth supplemented with tryptamine, indole-3-acetamide, and indole-3-acetonitrile, and incubated with shaking.

The liquid culture may then be centrifuged to collect the selected microbiome, washed in sterile buffer and re-suspended in buffer. The process may then be repeated.

The enriched functional microbiome is stored at −80° C. Large isolated colonies may be collected and stored at −80° C.

Constructing a ACC (1-aminocyclopropane-1-carboxylate) Deaminase Producing Functional Microbiome:

A sample of the extracted microbiome may be inoculated into a flask containing sterile nitrogen free Dworkin and Foster minimal media broth supplemented with 3 mM 1-amino-1-cyclopropane-carboxylate, and incubated with shaking. The liquid culture may then be centrifuged to collect the selected microbiome, washed in sterile buffer and resuspended in buffer. The process may then be repeated. The enriched functional microbiome is stored at −80° C. Large isolated colonies may be collected and stored at −80° C.

Constructing a Diazotrophic Functional Microbiome:

A sample of the extracted microbiome may be inoculated into a flask containing sterile nitrogen free Combined Carbon Source Media broth, and incubated with shaking. The liquid culture may then be centrifuged to collect the selected microbiome, washed in sterile buffer and resuspended in buffer. The enriched functional microbiome is stored at −80° C. Large isolated colonies may be collected and stored at −80° C.

Constructing a Abscisic Acid Functional Microbiome:

A sample of the extracted microbiome may be inoculated into a flask containing sterile carbon Dworkin and Foster minimal media broth supplemented with abscisic acid, and incubated by shaking. The liquid culture may then be centrifuged to collect the selected microbiome, washed in sterile buffer and resuspended in buffer. The enriched functional microbiome is stored at −80° C. Large isolated colonies may be collected and stored at −80° C.

Constructing a Organic Pollutant Degrading Functional Microbiome:

A sample of the extracted microbiome is inoculated into a flask containing sterile nitrogen free or carbon free or phosphate Dworkin and Foster minimal media broth supplemented with the specified compound, and incubated by shaking. The liquid culture is then centrifuged to collect the selected microbiome, washed in sterile buffer and resuspended in buffer. The enriched functional microbiome is stored at −80° C. Large isolated colonies are collected and stored at −80° C.

Constructing a Heavy Metal Resistance Functional Microbiome:

A sample of the extracted microbiome may be inoculated into a flask containing sterile Tris-gluconate broth supplemented with the specified heavy metal, and incubated by shaking. The liquid culture may then be centrifuged to collect the selected microbiome, washed in sterile buffer and resuspended in buffer. The enriched functional microbiome is stored at −80° C. Large isolated colonies may be collected and stored at −80° C.

Constructing a Salt Resistance Functional Microbiome:

A sample of the extracted microbiome may be inoculated into a flask containing sterile nutrient broth supplemented with the specified 0.6-3.8% NaCl, and incubated by shaking. The liquid culture may then be centrifuged to collect the selected microbiome, washed in sterile buffer and resuspended in buffer. The enriched functional microbiome is stored at −80° C. Large isolated colonies may be collected and stored at −80° C.

Constructing a Alkaline or Acidic Tolerant Functional Microbiome:

A sample of the extracted microbiome may be inoculated into a flask containing sterile nutrient broth with pH4 for acidic tolerant microbiome enrichment and pH9 for alkaline tolerance enrichment, and incubated by shaking. The liquid culture may then be centrifuged to collect the selected microbiome, washed in sterile buffer and resuspended in buffer. The enriched functional microbiome is stored at −80° C. Large isolated colonies may be collected and stored at −80° C.

Identification of the Isolates:

The purified isolates resulting from these rounds of selection may be identified through sequencing and bioinformatics analysis of their 16S rDNA gene. The isolates may be subjected to gram staining and further biochemical tests to establish their identification.

Characterisation of the Strains:

The isolates originating from the selection process above may be subjected to rapid high-through put screening assays, which involves a set of phenotypic and/or genotypic assays including, but not limited to, the following traits:

Preferable Plant-Growth Promotion Traits Include:

ACC deaminase activity, Inorganic phosphate solubilisation, Organic phosphate liberation, Indole-3-acetic acid production, Abscisic acid degradation, Diazotrophic activity, Exopolysaccharide production, production of 2,4 diacetylphloroglucinol, phenazine, phenylacetic acid, pyrrolnitrin and dimethylhexadecylamine.

Preferable Xenobiotic Degradation Traits Include:

Petroleum compounds (petrol, diesel, crude oil, lubrication oils), Polycyclic aromatic hydrocarbons (naphthalene, acenaphthylene, acenaphthene, fluorene, phenanthrene, anthracene, fluoranthene, pyrene, benz[a]anthracene, chrysene, benzo[b]fluoranthene, benzo[k]fluoranthene, benzo[a]pyrene, dibenz[a,h]anthracene, benzo[ghi]perylene and indeno[1,2,3-cd]pyrene), gamma-hexachlorocyclohexane (lindane), biocides: including parathion, malathion, methyl parathion, chlorpyrifos, diazinon, dichlorvos, phosmet, fenitrothion, tetrachlorvinphos, azamethiphos, Food Authority Organisation 1, and glyphosphate) atrazine, simazine, propazine and cyanazine), (2-methyl-4-chlorophenoxyacetic acid (MCPA), methylchlorophenoxypropionic acid (mecoprop), 2,4 Dichlorophenoxyacetic acid (2,4-D), 3-(3,4-dichlorophenyl)-1,1-dimethylurea (DCMU) aldrin, chlordane, DDT, dieldrin, hexachlorobenzene (HCB), heptachlor, endrin, and toxaphene, Bentazone, chlortoluron, cypermethrin, isoproturon, paraquat, pentachlorophenol, and 2,4,5-trichlorophenol), Nitroaromatics (including 2,4,6-trinitrotoluene, 1,3,5-Trinitrobenzene, 2-nitrophenol, 3-nitrophenol, 4-nitrophenol, 2,4-dinitrophenol, 2,5-dinitrophenol), organic solvents (acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxy-ethane (glyme, DME), dimethyl-formamide (DMF), dimethyl sulfoxide (DMSO), 1,4-dioxane, ethanol, ethyl acetate, ethylene, glycol, glycerin heptane, Hexamethylphosphoramide (HMPA), Hexamethylphosphorous triamide (HMPT)hexane methanol, methyl t-butyl ether (MTBE), methylene chloride, N-methyl-2-pyrrolidinone (NMP), nitromethane, pentane, Petroleum ether (ligroin),1-propanol, 2-propanol, pyridine, tetrahydrofuran (THF), toluene, triethyl amine, o-xylene, m-xylene p-xylene, trichlorethylene, Hexane, cyclohexane, Benzene, toluene, ethylbenzene), dioxins and furans, PCBs and cyanide.

Preferable Biocontrol Traits Include:

Phenyl acetic acid, 2,4 diacetylphloroglucinol and Phenazine

Preferable Heavy Metal Tolerance, Solubilisation or Immobilisation Traits Include:

Cadmium, Lead, Chromium, Nickel, Copper, Zinc, Cobalt, Mercury, Selenium. Bacterial isolates may be identified through DNA sequencing of the full 16S rDNA gene (˜1,500 bp) and subsequent bioinformatics analysis. Bacterial isolates selected for further product development may be of any suitable species. Factors for selection of bacterial isolates include the relatedness to human, animal, plant or environmental pathogens, only species from biosafety risk group 1 (non-pathogenic groups) may be selected for further product development. For example, the bacteria can be chosen from species including, but not limited to, Pseudomonas, Rhodococcus, Ralstonia, Alcaligenes, Streptomyces, Aeromonas, Rhizobia, Bradyrhizobium, Burkholderia, Achromobacter, Micrococcus, Bacillus, Azomonas, Derxia, Lignobacter, Rhodospirillium, Rhodo-pseudomonas, Herbaspillium, Acetobacter, Xanthobacter, Desulfovibrio, Clostridium, Actinomyces, Arthrobacter, Cladosporium, Staphylococcus, Acinetobacter, Xanthomonas, Sphingomonas, Enterobacter, Flavobacterium, Corynebacterium, Brevibacterium, Nocardia, Planococcus, Kocuria, Microbacterium, Paenibaccillus, Ochronobacterium, Serratia, Stenothrophomonas Azospillium, cellulomonas. Gluconacetobacter, Beijerinckia, Lactobacillus and Delftia.

Construction of a Functional Microbial Composition:

Depending on the stress conditions within the target application site, a collection of 1-1000 isolates per specific trait may be selected for inclusion in a constructed functional microbial composition. This selection process may be informed by data generated in the field-grown microbiome study showing the natural plant/crop microbiome community structure.

The core functional microbiome composition may consist of:
1-1000 ACC deaminase active strains;
1-1000 Indole-3-acetic acid producing strains;
1-1000 Phosphate solubilising strains;
1-1000 Diazotrophic strains;
1-1000 Abscisic acid degrading strains;
1-1000 Phenazine/2,4 DAPG producers, or combinations thereof.

In addition to this core composition, trait specific strains may be added depending on the nature of the stressors on a particular site. For example, if the site is contaminated with a particular organic compound, then 1-1000 strains with degradation ability of that compound would be included in the microbial consortia. Depending on the method of enrichment and selection used, substantially all of the strains within the composition may have the same general characteristic(s) (e.g. all strains may be resistant to a particular heavy metal or display salt tolerance), along with their own strain specific PGP characteristic(s).

Greenhouse Plant Growth Trials:

The target plant species may include any plant crop and non-crop species. Each of the selected isolates may be cultivated individually and may be mixed together. The enriched functional microbiome or the selected microbe or microbial combinations may then be applied to the test plants/crops in the greenhouse. This application may take the form of soil drenching or seed coating as liquid inoculum, liquid gel or solid gel (Carrageenan, alginate, polyacrylamide, agarose, cellulose, methylcellulose, gum Arabic etc.). Plants may be cultivated under conditions resembling as far as practically possible, the natural environmental conditions on the target site (including soil conditions, light conditions, moisture and temperature conditions). Plant growth parameters are plant species specific but may include plant height, total biomass, leaf/stem/root biomass leaf area index, nitrogen/phosphorus levels, number of flowers seed/fruit yield etc.

The invention includes the extraction and isolation of the microbiome associated with a single plant (or section of plants) from a particular site. The present invention also relates to a process of selecting, enriching and isolating the subset of a plant's or plants' and soil microbiome that expresses one or more plant growth promoting traits or other desired traits. The invention includes the process of creating a composition or consortium comprising one or more of these isolates, expressing specific traits, for application on agricultural food crops (cereals, vegetables, fruit) and non-food crop (bioenergy, fibre, pharmaceutical), and plants used in horticultural and phytoremediation applications and bioremediation application.

The constructed functional microbiome, selected microbe or microbial consortia of the invention are particularly suitable for plants cultivated in stressed soil (while also useful in non-stressed soils). Such stress conditions include, but are not limited to, drought, waterlogging, high salinity, low nutrient levels, contaminated with heavy metals, organic pollutants or plant pathogens/pests. The pollutant may be a hydrocarbon, especially but not limited to crude oil, petroleum or diesel, heavy lubricant oils, pesticides, herbicides, fungicides, volatile organic compounds, polychlorinated biphenyls, dioxins/furans, cyanide or polycyclic aromatic hydrocarbons. The pollutant may also include heavy metal, in particular, but not limited to, lead (Pb), chromium (Cr), arsenic (As), zinc (Zn), cadmium (Cd), copper (Cu), mercury (Hg), and nickel (Ni).

According to the present invention, the composition of the the constructed microbiome or microbial consortia includes bacterial and/or fungal strains that expresses one or more of the following traits; inorganic and/or organic phosphate and potassium liberation, ability to carry out nitrogen fixation (Diazotrophic activity), ability to produce plant growth hormones (indole-3-acetic acid, cytokinins, giberillins); ability to reduce the level of plant stress hormones (reduction in levels of ethylene through the degradation of 1-amino-1-cyclopropane carboxylate (ACC) due to the action of the bacterial enzyme ACC deaminase and the reduction in the level of abscisic acid in the plant roots), ability to produce action and 2,3-butanediol, can produce metabolites capable of inhibiting the growth of plant pathogens (viruses, bacteria, protozoans or fungi) and/or reduce plant attack by nematodes or insects, the ability to degrade toxic organic compounds in the soil including pesticides (insecticides, herbicides and fungicides), mineral oils, polycyclic aromatic hydrocarbons (PAHs), nitro aromatic compounds, halogenated and non-halogenated aromatics and aliphatic compounds, the ability to sequester, accumulate, solubilise or immobilise toxic heavy metals (including lead (Pb), cadmium (Cd), Arsenic (Ar), Chromium (Cr), Zinc (Zn), Copper (Cu), Nickel (Ni), Cobalt (Co) and Mercury (Hg) and/or be capable of surviving and growing in high saline conditions.

One of the advantages of the invention is that the entire microbial population associated with the plant and/or soil is collected, concentrated and used in the screening process to identify microorganisms with desired traits for the site. The most important advantage of the process is that it is site specific, i.e. for one particular site or crop that a specific functional microbiome, microbe or microbial consortia is selected, constructed and used in that site. By constructing multiple site-specific commercial products from multiple sites across the world, generic microbial consortia can be composed with desired traits for one or more plant by combining the most optimal isolated microbes from all sites. (FIG. 2,3)

### BRIEF DESCRIPTION OF THE DRAWINGS

FIG. 1 Schematic overview of the Constructed Functional Microbiome (CFM) isolation process; an embodiment for plant growth promotion for a target plant on a specific stressed or non-stressed site;

FIG. 2 An example of developing generic commercial products for a targeted plant by combining the best microbes from each site- to give specific microbes for a targeted plant; and

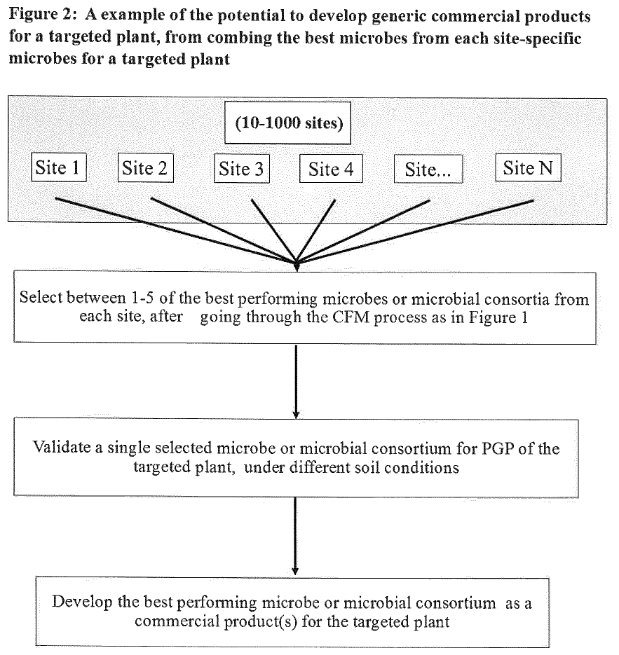

FIG. 3 An example of developing generic commercial products for multiple plant species under the same specific site conditions.

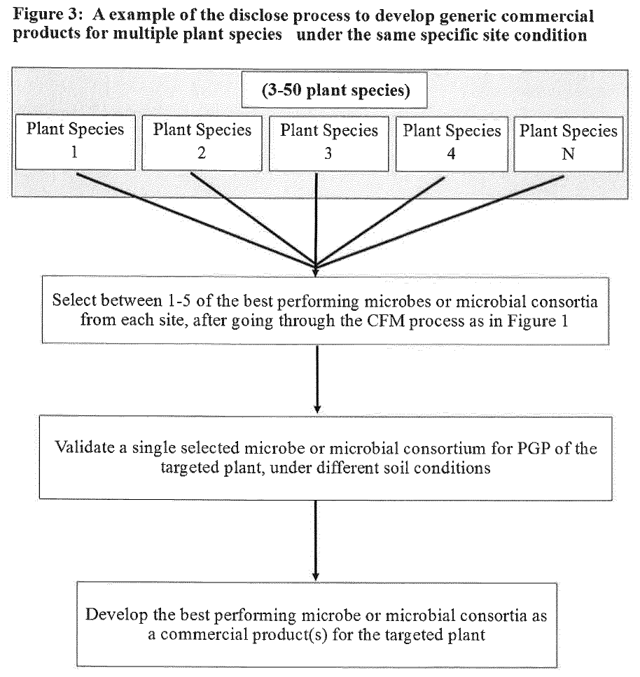

### DETAIL DESCRIPTION OF THE INVENTION

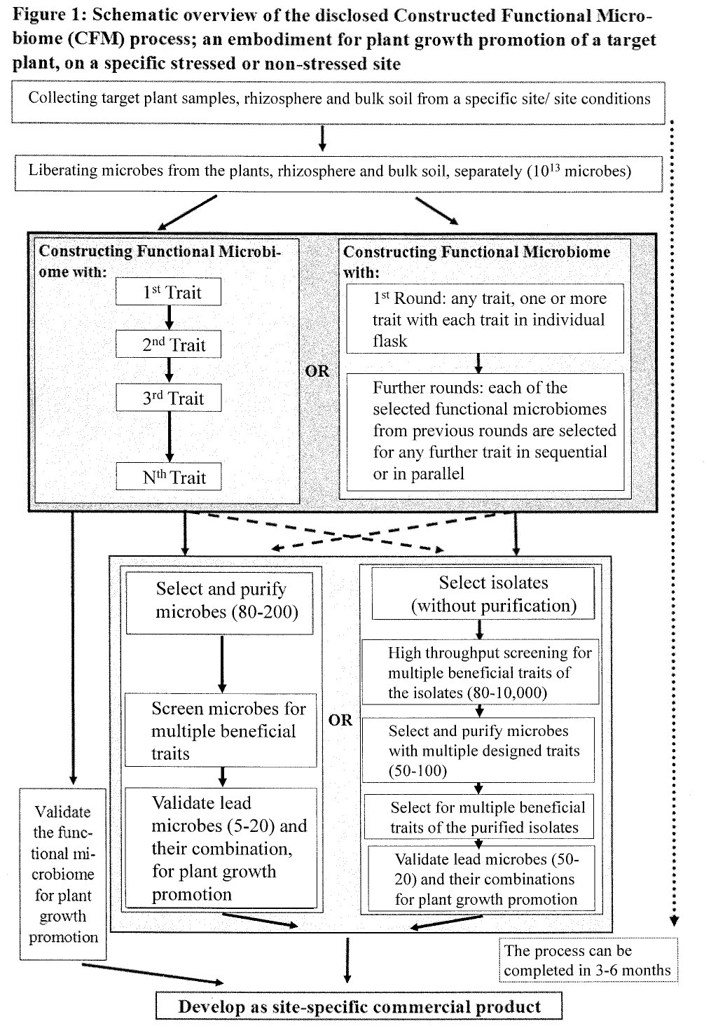

FIG. 1 shows an overview of the disclosed method of the invention.

According to one embodiment of the present invention, the total microbiome of targeted plant and soil from a target site (stressed or unstressed), is extracted from the plant material (FIG. 1). This involves homogenisation of the plant material in a sterile diluent. This diluent is designed to help liberate microbes from the plant material and to protect the microbes from changes in pH and possible anti-microbial compounds released from the plant and soil material after homogenisation. The homogenised mixture is then subjected to rigorous shaking (30-200 oscillation per minute) for 1-60 minutes to help liberate the microbes into the diluent. The plant material is removed by using low speed centrifugation (1000-6000 rpm). The supernatant containing all the liberated microbes is collected and is re-centrifuged at high speed (10,000-20,000 rpm) to concentrate and collect the microbes. The centrifuged pellet containing the plant microbiome is collected and re-suspended in sterile saline. This is then centrifuged again to remove plant derived nutrients and cell material, and the pellet re-suspended in sterile saline. This washing step is repeated once more. The final washed pellet is then re-suspended in 20 ml of saline containing 20% glycerol, and mixed thoroughly. 1 ml aliquots of this extracted microbiome are then aseptically transferred to 20 sterile 1.5 tubes, capped and stored at −80° C. until required. The soil microbiome is prepared as the plant microbiome extraction as above.

The extracted microbiome is subjected to selection and enrichment of its microbial members which possess specific plant growth promotion and/or biocontrol, and/or heavy metal tolerance/immobilisation/so lubilisation and/or organic pollutant degrading abilities and/or tolerance to high saline conditions. This selection and enrichment can be carried out in the following two processes, or any other combination of the processes.

1). The extracted microbiome can be inoculated into one or more of the following:

(a) Nitrogen free combined carbon broth;

(b) Nitrogen free Dworkin and Foster broth supplemented with tryptamine, indole-3-acetamide and indole-3-acetonitrile;

(c) Nitrogen free Dworkin and Foster broth supplemented with 1-amino-1-cyclopropane carboxylate;

(d) National Botanical Research Institute Phosphate growth media;

(e) Carbon free Dworkin and Foster broth supplemented with abscisic acid;

(f) Gluconate media supplemented with one or more of lead, cadmium, nickel, zinc, copper, cobalt, mercury;

(g) Nutrient broth containing 6% NaCl;

(h) Nutrient broth containing with pH4 or pH9;

(i) Carbon free Dworkin and Foster broth supplemented with any organic pollutant;

(j) Nitrogen free Dworkin and Foster broth supplemented with any organic pollutant;

(k) Phosphate free National Botanical Research Institute Phosphate growth media

Each may be supplemented with any organic pollutant.

Each of these primary selection and enrichment cultures are incubated for 1-14 days, at 0-200 rpm and 10-30° C. After the designated incubation time the entire microbiome of selected and enriched cells are collected by high speed centrifugation, washed twice in sterile saline, resuspended in 5 ml sterile saline and 1 ml used to inoculate another flask containing identical growth media to the primary culture. Each of these secondary selection and enrichment cultures are incubated for 1-14 days, at 0-200 rpm and 10-30° C. After the designated incubation time the entire microbiome of selected and enriched cells is collected by high speed centrifugation, washed twice in sterile saline, resuspended in sterile saline. This selection process can go through another 1-9 rounds of enrichments with media identical to the primary culture. After the final round of enrichment, the washed pellet is then re-suspended in 20 ml of saline containing 20% glycerol, and mixed thoroughly. 1 ml aliquots of this extracted microbiome are then aseptically transferred to 20 sterile 1.5 tubes, capped and stored at −80° C. 1 ml of this selected microbiome is used to prepare a serial dilution from 10⁻¹-10⁻⁷. Samples of these dilutions are plated onto identical growth media used in the primary and secondary selection culture but solidified with agar. Each of these agar plates are incubated for 1-14 days at 10-30° C. After the designated incubation time the plates are examined for growth of individual colonies.

Using a high-through put robotic colony picker, each of the colonies is picked and transferred to a single well of a 96 well microtitre plate containing nutrient broth. The plates are incubated for 24 hours at 150 rpm 20-30° C. Colonies that show good growth in the wells are selected for storage and further characterisation.

2) The extracted microbiome is then inoculated into one of the following media:

(a) Nitrogen free combined carbon broth;

(b) Nitrogen free Dworkin and Foster broth supplemented with tryptamine, indole-3-acetamide and indole-3-acetonitrile; or

(c) Nitrogen free Dworkin and Foster broth supplemented with 1-amino-1-cyclopropane carboxylate; or

(d) National Botanical Research Institute Phosphate growth media; or

(e) Carbon free Dworkin and Foster broth supplemented with abscisic acid; or

(f) Gluconate media supplemented with one or more of lead, cadmium, nickel, zinc, copper, cobalt, mercury; or

(g) Nutrient broth containing 6% NaCl; or

(h) Carbon free Dworkin and Foster broth supplemented with any organic pollutant; or

(i) Nitrogen free Dworkin and Foster broth supplemented with any organic pollutant; or

(j) Phosphate free National Botanical Research Institute Phosphate growth media supplemented with any organic pollutant.

This primary selection and enrichment culture is incubated for 1-14 days, at 0-200 rpm and 10-30° C. After the designated incubation time the entire microbiome of selected and enriched cells is collected by high speed centrifugation, washed twice in sterile saline, resuspended in 5 ml sterile saline. This primary enriched culture can either be plated out on selective solid medium for isolating colonies or go through another 1-9 rounds of sequential enrichments with different medium in each round. The medium may be either
(a) Nitrogen free combined carbon broth; or
(b) Nitrogen free Dworkin and Foster broth supplemented with tryptamine, indole-3-acetamide and indole-3-acetonitrile; or
(c) Nitrogen free Dworkin and Foster broth supplemented with 1-amino-1-cyclopropane carboxylate; or
(d) National Botanical Research Institute Phosphate growth media; or
(e) Carbon free Dworkin and Foster broth supplemented with abscisic acid; or
(f) Gluconate media supplemented with one or more of lead, cadmium, nickel, zinc, copper, cobalt, mercury; or
(g) Nutrient broth containing 6% NaCl; or
(h) Nutrient broth containing with pH4 or pH9; and/or
(i) Carbon free Dworkin and Foster broth supplemented with any organic pollutant; or
(j) Nitrogen free Dworkin and Foster broth supplemented with any organic pollutant; or
(k) Phosphate free National Botanical Research Institute Phosphate growth media supplemented with any organic pollutant.

Each of these selection and enrichment cultures are incubated for 1-14 days, at 0-200 rpm and 10-30° C. After the designated incubation time, the entire microbiome of selected and enriched cells of each round of enrichment are collected by high speed centrifugation, washed twice in sterile saline, resuspended in 5 ml sterile saline. The washed pellet is then re-suspended in 20 ml of saline containing 20% glycerol, and mixed thoroughly. 1 ml of the aliquots of the extracted microbiome are then aseptically transferred to 20 sterile 1.5 tubes, capped and stored at −80° C. 1 ml of final selected functional microbiome is used to prepare a serial dilution from 10⁻¹-10⁻⁷. Samples of these dilutions are plated onto identical growth media used in the tertiary selection culture but solidified with agar. Each of these agar plates are incubated for 1-14 days at 10-30° C. After the designated incubation time the plates are examined for growth of individual colonies. Using a high-through put robotic colony picker, each of the colonies are picked and transferred to a single well of a 96 well microtitre plate containing nutrient broth. The plates are incubated for 24 hours at 150 rpm 20-30° C. Colonies that show good growth in the wells are selected for storage and further characterisation.

The isolates will be subjected to high throughput screening assays to identify other desired traits. These screening assays will identify the following traits as examples but not limited within individual isolates;
ACC deaminase activity
Abscisic acid degradation
Phosphate solubilisation
IAA production
Diazotrophic activity
Organic pollutant degrading ability: VOCs, PAHs PCBs, Crude oil, nitroaromatics, pesticides, cyanide)
Heavy metal tolerance/solubilisation/immobilisation

Isolates showing strong activity of one or more of the above traits are purified 3-5 times. subjected to high throughput screening assays to determine desired traits. These screening assays will identify the following traits as examples but not limited within individual isolates;
ACC deaminase activity
Abscisic acid degradation
Phosphate solubilisation
IAA production
Diazotrophic activity
Organic pollutant degrading ability: VOCs, PAHs PCBs, Crude oil, nitroaromatics, pesticides, cyanide)
Heavy metal tolerance/so lubilisation/immobilisation

The purified isolates are then subjected to a minimum of Gram staining, endospore staining and identification based on sequencing and bioinformatics analysis of their full 16S rDNA gene. The enriched functional microbiome, microbe or microbial consortia can be selected to apply for promoting plant growth, plant and soil health, food safety and bioremediation.

### EXAMPLES

The following specific examples illustrate the process and efficacy of this invention, but they should not be construed as limiting the scope of the invention. Reasonable variations and modifications are possible within the scope of this disclosure without departing from the spirit and scope of this invention.

While this invention has been described in detail with reference to certain preferred embodiments, it should be appreciated that the present invention is not limited to those precise embodiments rather, in view of the present disclosure, which describes the current best mode for practicing the invention, many modifications and variations would present themselves to those skilled in the art without departing from the scope and spirit of this invention. This invention is claims.

### Example 1: Constructing a Site-Specific Functional Microbiome to Screen and Identify Microbes with Beneficial Traits for Rice (Oryza sativa) Plants Growing on Cadmium Impacted Agricultural Soil

Cadmium impacted arable land is a major problem in China. It impacts the growth of the rice plant and poses a treat to food safety by the accumulation of cadmium within the rice. It would be desirable to apply the present invention to develop site-specific functional microbiomes, microorganisms or consortia to increase the rice crop yield and reduce the accumulation of cadmium within the rice to protect food safety and human health.

Rice (Oryza sativa) plants and bulk soil were collected from 10 different rice fields in Hunan province, China. The complete plants were taken, including the rhizosphere, rhizoplane, endosphere and phyllosphere, to harbour microbes with numerous beneficial traits. The objective of this procedure is to extract, collect and store as much of the plant microbiome as possible from plant samples and bulk soil sourced from the impacted agricultural land. This total plant microbiome will consist of microbes originating from various parts of the plant including the rhizosphere, rhizoplane, endosphere and phyllosphere. Soil and whole plant samples sourced from impacted land were used as a source of functional microbiomes for selective enrichment of plant growth promoting and heavy metal tolerant microbes.

Procedure: Excess soil was removed from plant roots and stored for future analysis. The plant was homogenised in sterile Phosphate buffer saline with 0.05% Tween 20, using a sterile blender. The homogenised plant sample was transferred to 250 ml centrifuge tubes and shaken in a wrist action shaker for 10 mins at 4° C. Plant material was removed from the samples by gentle centrifugation and the supernatant was collected in a fresh centrifuge tube. The bacterial cells in the supernatant were collected by centrifuging at high speed. The bacterial pellet was washed in triplicate and subsequently re-suspended in sterile ringers. The microbes were also collected from the bulk soil samples. 1 ml aliquots of the resulting microbiome were stored in 90% glycerol at −80° C. until required.

Results: Three type of plant microbiome extractions were constructed as MGSAMP005, MGSAMP006 and MGSAMP008; and three soil microbiome extractions were constructed as MGSAMPBS001, MGSAMPBS002 and MGSAMP010. There extracted microbiomes were stored in −80° C. All extracted microbiomes were enriched with a first trait to construct a heavy metal tolerant functional microbiome. Subsequently the heavy metal tolerant functional microbiomes were enriched with other beneficial traits to construct functional microbiomes with multiple traits detailed below.

Constructing Heavy Metal Resistant Functional Microbiome—1^{st} Trait

Principle: Most metal ions have to enter the bacterial cell in order to have a physiological or toxic effect. Many divalent metal cations (e.g. Mn2+, Fe2+, Co2+, Ni2+, Cu2+ and Zn2+) are structurally very similar. Also, the structure of oxyanions such as chromate resembles that of sulfate, and the same is true for arsenate and phosphate. Thus to be able to differentiate between structurally very similar metal ions, the microbial uptake systems have to be tightly controlled. Microorganisms use fast and unspecific uptake systems driven by the chemiosmotic gradient across the cytoplasmic membrane of bacteria. These uptake systems are constitutively expressed and thus, they lead to the accumulation of heavy metal ions within the microbial cell. Since high concentrations of heavy metal ions within the microbial cells are very toxic, microorganisms have been forced to develop metal-ion homeostasis factors or metal-resistance determinants. These resistance determinants encode proteins which play a role in detoxification mechanisms for the survival of microorganisms in heavy-metal contaminated environments. Another type of uptake system has high substrate specificity, is slower, and often uses ATP hydrolysis as the energy source. As opposed to constitutively expressed unspecific uptake systems, ATP-dependent uptake systems are inducible. Inside the cell, the toxicity of heavy metal ions may occur through the displacement of essential metals from their native binding sites or through ligand interactions. Heavy metal cations especially those with high atomic numbers, e.g. Hg2+, Cd2+ and Ag+, tend to bind SH groups. Growth media with high phosphate contents can interfere with the toxicity of metals on cell physiology, either by competing for uptake systems or by chemically reacting with the metals and forming insoluble precipitates which reduce the bioavailability of the metal. For this reason this heavy metal enrichment assay utilises low nutrient Tris-gluconate broth.

Procedure: 1 ml of each of the plant microbiomes were inoculated into Tris-gluconate broth supplemented with 2 mM Cd, Zn and Pb (CZL). 1 ml of each of the soil microbiomes were inoculated into the same media. The pH of the growth media was adjusted to 6.5±0.2 at 25° C. The enrichment was incubated for 7 days at 27° C. and 100 rpm. Following incubation, the culture was transferred to a 250 ml tube and centrifuged at 20,000 rpm, 4° C. for 20 mins. The supernatant was removed and the bacterial pellet was washed in triplicate with sterile ringers. After the final wash the bacterial pellet was re-suspended in ringers, dispensed into 1 ml aliquots and stored at −70° C. in 90% glycerol. 1 ml of the primary enrichment was retained and used to inoculate the second enrichment. 1 ml of the primary enrich microbiome was inoculated into fresh Tris-gluconate broth with CZL and incubated as described above. The secondary enrichment microbiome was collected and stored as detailed above with 1 ml of this enrichment being retained for isolation of microbes with heavy metal tolerance.

1 ml of the the enriched functional microbiome was serially diluted down to 10⁻⁶ and plated out onto Tris-gluconate agar with 2 mM CZL. Plates were incubated for 5 days at 20° C. A Qpix colony picker was used to select colonies that displayed large zones of clearing and significant discolouration and transfered into a 96 well microtitre plate containing nutrient broth, plates were incubated for 5 days at 20° C. Plates were examined for growth and the Qpix colony picker was used to create a compressed library of the actively growing cultures. Library plates were stored in triplicate at −70° C. in 90% glycerol until required.

Constructing ACC Deaminase Producing Functional Microbiomes—2^{nd} Trait

Principle: The enzyme 1-aminocyclopropane-1-carboxylate (ACC) deaminase promotes plant growth by sequestering and cleaving plant-produced ACC thereby lowering the level of ethylene in the plant. Decreased ethylene levels allow the plant to be more resistant to a wide variety of environmental stresses. It is known that less than 10% of soil/plant microbes possess ACC deaminase activity. The objective of this procedure is to select or/and to enrich microbes from the extracted plant microbial community that possess ACC deaminase activity. The assay is based on the principal that cleavage of ACC by ACC deaminase results in the production of α-ketobutyrate and ammonia. These two compounds can then be utilised by the microbes as a carbon and nitrogen source. When grown in a culture media without nitrogen, but supplemented with ACC only those microbes possessing ACC deaminase activity will be able to actively grow (although there are microbes that have alternative deamination enzymes may also be present).

Procedure: 1 ml of the heavy metal tolerant plant functional microbiome were inoculated into DF growth media containing ACC hydrochloride as the sole nitrogen source. 1 ml of the heavy metal tolerant soil functional microbiome were inoculated into the same media. The pH of the growth media was adjusted to 7.2±0.2 at 25° C. The enrichment was incubated for 7 days at 27° C. and 100 rpm. Following incubation, the culture was transferred to a 250 ml tube and centrifuged at 20,000 rpm, 4° C. for 20 mins. The supernatant was removed and the bacterial pellet was washed in triplicate with sterile ringers. After the final wash the bacterial pellet was re-suspended in ringers, dispensed into 1 ml aliquots and stored at −70° C. in 90% glycerol. 1 ml of the this enrichment was retained and used to inoculate the next round of enrichment.

1 ml of the enriched functional microbiome was serially diluted down to 10⁻⁶ and plated onto DF agar with ACC. Plates were incubated for 5 days at 20° C. A Qpix colony picker was used to select colonies that displayed large zones of clearing and significant discolouration and transfered into a 96 well microtitre plate containing nutrient broth, plates were incubated for 5 days at 20° C. Plates were examined for growth and the Qpix colony picker was used to create a compressed library of the actively growing cultures. Library plates were stored in triplicate at −70° C. in 90% glycerol until required.

Constructing Indole-3-Acetic Acid Producing Functional Microbiomes—3^{rd} Trait

Principle: Indole acetic acid (IAA) is one of the most physiologically active auxins in plants. It stimulates the production of longer roots with increased number of root hairs and root laterals that are involved in nutrient uptake, promotes cell elongation and regulates cell osmotic potential. IAA is a common product of L-tryptophan metabolism produced by several microorganisms including Plant Growth-Promoting bacteria (PGPB). There are a number of different IAA biosynthesis pathways found in PGPB and a bacterial cell may contain multiple pathways. This enrichment assay is based on the fact that in many of these IAA production pathways there are steps that result in the production of ammonia. This ammonia can be utilised by the microbes as a nitrogen source. When grown in a culture media without nitrogen, but supplemented with various intermediates in the IAA pathways, only those microbes possessing actively expressed IAA pathway genes will be able to grow (although there may be microbes present that have alternative enzymes that may also release ammonia or degrade these intermediate compounds). In this enrichment assay three IAA intermediates are utilised to select for and enrich microbes with one (or more) of three different IAA biosynthesis pathways. Each of these compounds is subsequently converted into IAA or IAA precursors, with the release of ammonia.

Procedure: 1 ml of the heavy metal-ACC deaminase enriched plant functional microbiome were inoculated into DF growth media containing a mixture of IAA intermediates as the sole nitrogen source. 1 ml of the heavy metal-ACC deaminase enriched soil functional microbiome were inoculated into the same media. The intermediate solution consisted of Tryptamine, Indole-3-acetamide and Indole-3-acetonitrile, the final concentration of the intermediate mixture in the media was 4.5 mM. The pH of the growth media was adjusted to 7.2±0.2 at 25° C. The enrichment was incubated for 7 days at 27° C. and 100 rpm. Following incubation, the culture was transferred to a 250 ml tube and centrifuged at 20,000 rpm, 4° C. for 20 mins. The supernatant was removed and the bacterial pellet was washed in triplicate with sterile ringers. After the final wash the bacterial pellet was re-suspended in ringers, dispensed in 1 ml aliquots and stored at −70° C. in 90% glycerol. 1 ml of the this enrichment was retained and used to inoculate the next round of enrichment.

1 ml of the the enriched functional microbiome was serially diluted down to 10⁻⁶ and plated out onto DF agar with IAA intermediates. Plates were incubated for 5 days at 20° C. A Qpix colony picker was used to select colonies that displayed large zones of clearing and significant discolouration and transfer into a 96 well microtitre plate containing nutrient broth, plates were incubated for 5 days at 20° C. Plates were examined for growth and the Qpix colony picker was used to create a compressed library of the actively growing cultures. Library plates were stored in triplicate at −70° C. in 90% glycerol until required.

Constructing Abscisic Acid Producing Functional Microbiomes—4^{th} Trait

Principle: The plant hormone abscisic acid (ABA) is the major player in mediating the adaptation of the plant to stress. Abscisic acid is produced in the roots of plants that are exposed to stresses such as drought and toxic chemicals. From the roots it is transported by the transpiration stream up into the leaves of the plant, where it binds to receptors on the guard cells of the stomata. This causes the guard cells to lose tugor pressure resulting in the closure of the stomata and a reduction in the transpiration rate of the plant. Since 90% of the water uptake by a plant is lost in evapotranspiration, this process allows plants to conserve their water supply or reduce the uptake of dissolved toxic pollutants. However, it also reduces nutrient up-take and so limits plant growth. Abscisic acid is also involved in other plant responses to stress such as leaf abscission. The reduction of abscisic acid in the root has been shown to result in increased plant growth. This selection and enrichment assay is based on the use of abscisic acid as a sole carbon source by abscisic acid degrading microbes. When grown in a culture media without carbon, but supplemented with abscisic acid only those microbes possessing actively expressed abscisic acid degradation genes will be able to grow.

Procedure: 1 ml of the heavy metal-ACC deaminase-IAA enriched plant functional microbiome were inoculated into DF growth media supplemented with 10 mg/l ABA as the sole carbon source. 1 ml of heavy metal-ACC deaminase-IAA enriched soil functional microbiome were inoculated into the same media. The enrichment was incubated for 7 days at 27° C. and 100 rpm. Following incubation, the culture was transferred to a 250 ml centrifuge tube and centrifuged at 20,000 rpm, 4° C. for 20 mins. The supernatant was removed and the bacterial pellet was washed in triplicate with sterile ringers. After the final wash the bacterial pellet was re-suspended in ringers, dispensed into 1 ml aliquots and stored at −70° C. in 90% glycerol. 1 ml was retained and used to inoculate the next round enrichment.

1 ml of the the enriched functional microbiome was serially diluted down to 10⁻⁶ and plated out onto DF agar with ABA. Plates were incubated for 5 days at 20° C. A Qpix colony picker was used to select colonies and transfer into a 96 well microtitre plate containing nutrient broth, plates were incubated for 5 days at 20° C. Plates were examined for growth and the Qpix colony picker was used to create a compressed library of the actively growing cultures. Library plates were stored in triplicate at −70° C. in 90% glycerol until required.

Constructing Phosphate Solubilising Functional Microbiomes—5^{th} Trait

Principle :The objective of this procedure is to select or enrich members of the extracted plant microbial community that have strong inorganic phosphate solubilising ability. The assay assumes that in the absence of soluble phosphate, only those microbes possessing the ability to solubilise inorganic sources of phosphate will survive and be enriched in the growth media. The activity of these phosphate-solubilising bacteria is likely to release phosphate into the media, which will support the growth of non-phosphate solubilsers. However, their populations are likely to remain low in comparison with effective solubilisers. The presence of iron and aluminium compounds, coupled with an alkaline pH is designed to limit the time that the liberated phosphate remains soluble in the media, thereby reducing the growth of non-solubilisers.

Procedure: 1 ml of the heavy metal-ACC deaminase-IAA-ABA enriched plant functional microbiome were inoculated into National Botanical Research Institute's Phosphate (NBRIP) growth media containing tricalcium phosphate as the sole phosphate source. 1 ml of heavy metal-ACC deaminase-IAA enriched soil functional microbiome were inoculated into the same media. The pH of the growth media was adjusted to 8.0±0.2 at 25° C. The enrichment was incubated for 7 days at 27° C. and 100 rpm. Following incubation, the culture was transferred to a 250 ml centrifuge tube, carefully avoiding the insoluble phosphate powder, and centrifuged at 20,000 rpm, 4° C. for 20 mins. The supernatant was removed and the bacterial pellet was washed in triplicate with sterile ringers. After the final wash the bacterial pellet was re-suspended in ringers, dispensed into 1 ml aliquots and stored at −70° C. in 90% glycerol. 1 ml of the this enrichment was retained and used to inoculate the next round of enrichment.

1 ml of the the enriched functional microbiome was serially diluted down to 10⁻⁶ and plated out onto NBRIP agar with 5 mg/L bromophenol blue. Plates were incubated for 5 days at 20° C. A Qpix colony picker was used to select colonies that displayed large zones of clearing and significant discolouration and transfer into a 96 well microtitre plate containing nutrient broth, plates were incubated for 5 days at 20° C. Plates were examined for growth and the Qpix colony picker was used to create a compressed library of the actively growing cultures. Library plates were stored in triplicate at −70° C. in 90% glycerol until required.

Constructing Diazotrophic Functional Microbiomes—6^{th} Trait

Principle: Nitrogen is an essential element in plant development and a limiting factor in plant growth. It represents about 2% of the total plant dry matter that enters the food chain. Nevertheless, plants cannot directly access dinitrogen gas, which makes up about 80% of the atmosphere. Plants absorb the available nitrogen in the soil through their roots in the form of ammonium and nitrates. Only some prokaryotes are able to use atmospheric nitrogen through a process known as biological nitrogen fixation (BNF), which is the conversion of atmospheric N2 to NH3, a form that can be used by plants. Diazotrophs are the bacteria responsible for nitrogen, they encode nitrogenase, the enzyme complex that catalyses the conversion of N2 gas to ammonia. This enrichment assay utilises a carbon rich, nitrogen free media and cultivation under anoxic conditions to select for and enrich nitrogen fixing microbes from the plant microbiome extract.

Procedure: 1 ml of the heavy metal-ACC deaminase-IAA-phosphate enriched plant functional microbiome were inoculated into Combined Carbon Source (CCM) growth media containing 5 μg/ml biotin. 1 ml of the heavy metal-ACC deaminase-IAA-phosphate enriched soil functional microbiome were inoculated into the same media. The pH of the growth media was adjusted to 7.2±0.2 at 25° C. The enrichment was incubated in an air tight container for 7 days at 27° C. and 100 rpm. Following incubation, the culture was transferred to a 250 ml centrifuge tube and centrifuged at 20,000 rpm, 4° C. for 20 mins. The supernatant was removed and the bacterial pellet was washed in triplicate with sterile ringers. After the final wash the bacterial pellet was re-suspended in ringers, dispensed into 1 ml aliquots and stored at −70° C. in 90% glycerol.

1 ml of the the enriched functional microbiome was serially diluted down to 10⁻⁶ and plated out onto CCM agar. Plates were incubated for 5 days at 20° C. A Qpix colony picker was used to select colonies and transfer into a 96 well microtitre plate containing nutrient broth, plates were incubated for 5 days at 20° C. Plates were examined for growth and the Qpix colony picker was used to create a compressed library of the actively growing cultures. Library plates were stored in triplicate at −70° C. in 90% glycerol until required.

Results

From the three plant and three soil microbiome extractions, functional microbiomes were constructed and stored from each round of the enrichment. A total of 1400 microbes were isolated without purification and are detailed in Tables 1 to Table 6.

TABLE 1
Bacterial microbiomes from site MGSAMP005,
multiple traits and heavy metal tolerance

| | Plant growth promotion traits | | | | | | |
|---|---|---|---|---|---|---|---|
| | | ACC | | | Heavy Metal | | |
| MG Isolate | Abscisic Acid | deaminase | Nitrogen | Phosphate | tolerance (2 mM) | | |
| ID | Degradation | activity | fixation | solubilisation | Cd | Zn | Pb |
| ABA001 | + | − | + | + | − | − | − |
| ABA002 | + | − | + | + | − | − | − |
| ABA003 | + | − | + | + | − | − | − |
| ABA004 | + | − | + | + | − | − | − |
| ABA005 | + | − | + | + | − | − | − |
| ABA006 | + | − | + | + | + | + | + |
| ABA007 | + | − | + | + | + | + | + |
| ABA008 | + | − | + | + | + | + | + |
| ABA009 | + | − | + | + | + | + | + |
| ABA010 | + | − | + | + | − | − | − |
| ABA011 | + | − | + | + | − | − | − |
| ABA012 | + | − | + | + | − | − | − |
| ABA013 | + | − | + | + | + | + | + |
| ABA014 | + | − | + | + | + | + | + |
| ABA015 | + | − | + | + | + | + | + |
| ABA016 | + | − | + | + | + | + | + |
| ABA017 | + | − | + | + | − | − | − |
| ABA018 | + | − | + | − | − | − | − |
| ABA019 | + | − | + | − | + | + | + |
| ABA020 | + | − | + | − | + | + | + |
| ABA021 | + | − | + | + | + | + | + |
| ABA022 | + | − | + | + | + | + | + |
| ABA023 | + | − | + | + | | | |
| ABA024 | + | − | + | + | + | + | + |
| ABA025 | + | − | + | + | + | + | + |
| ABA026 | + | − | + | − | − | − | − |
| ABA027 | + | − | + | + | − | − | − |
| ABA028 | + | − | + | − | + | + | + |
| ABA029 | + | − | + | − | − | − | − |
| ABA030 | + | − | + | − | − | − | − |
| ABA031 | + | − | + | + | − | − | − |
| ABA032 | + | − | + | + | − | − | − |
| ABA033 | + | − | + | + | − | − | − |
| ABA034 | + | − | + | + | − | − | − |
| ABA035 | + | − | + | + | − | − | − |
| ABA036 | + | − | + | − | − | − | − |
| ABA037 | + | − | + | − | − | − | − |
| ABA038 | + | − | + | + | − | − | − |
| ABA039 | + | − | + | + | − | − | − |
| ABA040 | + | − | + | + | + | + | + |
| ABA041 | + | − | + | + | + | + | + |
| ABA042 | + | − | + | + | − | − | − |
| ABA043 | + | − | + | + | − | − | − |
| ABA044 | + | − | + | − | − | − | − |
| ABA045 | + | − | + | − | − | − | − |
| ABA046 | + | − | + | + | − | − | − |
| ABA047 | + | − | + | + | + | + | + |
| ABA048 | + | − | + | + | + | + | + |
| ABA049 | + | − | + | + | + | + | + |
| ABA050 | + | − | + | + | − | − | − |
| ABA051 | + | − | + | + | − | − | − |
| ABA052 | + | − | + | − | − | − | − |
| ABA053 | + | − | + | + | − | − | − |
| ABA054 | + | − | + | + | − | − | − |
| ABA055 | + | − | + | + | − | − | − |
| ABA056 | + | − | + | + | + | + | + |
| ABA057 | + | − | + | + | + | + | + |
| ABA058 | + | − | + | + | − | − | − |
| ABA059 | + | − | + | + | − | − | − |
| ABA060 | + | − | + | + | − | − | − |
| ABA061 | + | − | + | + | − | − | − |
| ABA062 | + | − | + | + | − | − | − |
| ABA063 | + | − | + | + | − | − | − |
| ABA064 | + | − | + | + | + | + | + |
| ABA065 | + | − | + | + | + | + | + |
| ABA066 | + | − | + | + | − | − | − |
| ABA067 | + | − | + | + | − | − | − |
| ABA068 | + | − | + | + | − | − | − |
| ABA069 | + | − | + | − | − | − | − |
| ABA070 | + | − | + | + | − | − | − |
| ABA071 | + | − | + | + | − | − | − |
| ABA072 | + | − | + | + | − | − | − |
| ABA073 | + | − | + | + | − | − | − |
| ABA074 | + | − | + | + | − | − | − |
| ABA075 | + | − | + | + | − | − | − |
| ABA076 | + | − | + | + | − | − | − |
| ABA077 | + | − | + | + | − | − | − |
| ABA078 | + | − | + | + | − | − | − |
| ABA079 | + | − | + | + | − | − | − |
| ABA080 | + | − | + | + | − | − | − |
| ABA081 | + | − | − | + | + | + | + |
| ABA082 | + | − | + | + | − | − | − |
| ABA083 | + | − | + | + | − | − | − |
| ABA084 | + | − | + | + | − | − | − |
| ABA085 | + | − | + | + | − | − | − |
| ABA086 | + | − | + | + | + | + | + |
| ABA087 | + | − | + | + | + | + | + |
| ABA088 | + | − | + | + | − | − | − |
| ABA089 | + | − | + | + | − | − | − |
| ABA090 | + | − | + | + | − | − | − |
| ABA091 | + | − | + | + | − | − | − |
| ABA092 | + | − | + | + | − | − | − |
| CZL093 | − | + | + | − | + | + | + |
| CZL094 | − | + | + | − | + | + | + |
| CZL095 | − | + | + | − | + | + | + |
| CZL096 | − | + | + | − | + | + | + |
| CZL097 | − | + | + | − | + | + | + |
| CZL098 | − | + | + | + | + | + | + |
| CZL099 | − | + | + | + | + | + | + |
| CZL100 | − | − | − | + | + | + | + |
| CZL101 | − | + | + | + | + | + | + |
| CZL102 | − | + | − | − | + | + | + |
| CZL103 | − | + | − | − | + | + | + |
| CZL104 | − | + | + | + | + | + | + |
| CZL105 | − | + | + | + | + | + | + |
| CZL106 | − | + | + | + | + | + | + |
| CZL107 | − | + | + | + | + | + | + |
| CZL108 | − | + | − | + | + | + | + |
| CZL109 | − | + | + | + | + | + | + |
| CZL110 | − | + | + | + | + | + | + |
| CZL111 | − | + | + | + | + | + | + |
| CZL112 | − | + | + | + | + | + | + |
| CZL113 | − | − | − | + | + | + | + |
| CZL114 | − | + | + | + | + | + | + |
| CZL115 | − | + | + | + | + | + | + |
| CZL116 | − | + | + | − | + | + | + |
| CZL117 | − | + | + | + | + | + | + |
| CZL118 | − | − | + | − | + | + | + |
| CZL119 | − | + | + | + | + | + | + |
| CZL120 | − | + | + | − | + | + | + |
| CZL121 | − | − | − | + | + | + | + |
| CZL122 | − | + | + | + | + | + | + |
| CZL123 | − | + | + | + | + | + | + |
| CZL124 | − | + | | + | + | + | + |
| CZL125 | − | + | + | − | + | + | + |
| CZL126 | − | + | − | − | + | + | + |
| CZL127 | − | + | + | + | + | + | + |
| CZL128 | − | + | + | + | + | + | + |
| CZL129 | − | + | + | + | + | + | + |
| CZL130 | − | + | + | + | + | + | + |
| CZL131 | − | + | + | + | + | + | + |
| CZL132 | − | + | + | + | + | + | + |
| CZL133 | − | + | + | + | + | + | + |
| CZL134 | − | + | + | + | + | + | + |
| CZL135 | − | + | + | + | + | + | + |
| CZL266 | + | + | + | + | + | + | + |
| CZL267 | − | + | − | + | + | + | + |
| CZL268 | − | + | − | + | + | + | + |
| CZL269 | − | − | − | − | + | + | + |
| CZL270 | − | + | − | + | + | + | + |
| CZL271 | − | − | − | − | + | + | + |
| Diazo136 | − | + | + | − | − | − | − |
| Diazo137 | − | − | + | − | − | − | − |
| Diazo138 | + | + | + | + | − | − | − |
| Diazo139 | + | + | + | + | − | − | − |
| Diazo140 | + | + | + | + | + | + | + |
| Diazo141 | − | + | + | + | + | + | + |
| Diazo142 | − | + | + | + | | | |
| Diazo143 | − | − | + | − | + | + | + |
| Diazo144 | − | + | + | − | − | − | − |
| Diazo145 | − | + | + | + | + | + | + |
| Diazo146 | + | + | + | + | − | − | − |
| Diazo147 | + | + | + | + | + | + | + |
| Diazo148 | − | + | + | − | + | + | + |
| Diazo149 | + | + | + | − | + | + | + |
| Diazo150 | + | + | + | + | + | + | + |
| Diazo151 | − | − | + | − | − | − | − |
| Diazo152 | − | − | + | − | + | + | + |
| Diazo153 | − | − | + | − | + | + | + |
| Diazo154 | + | + | + | + | + | + | + |
| Diazo155 | + | + | + | + | + | + | + |
| Diazo156 | − | + | + | − | + | + | + |
| Diazo157 | − | + | + | − | + | + | + |
| Diazo158 | − | − | + | − | + | + | + |
| Diazo159 | − | − | + | − | + | + | + |
| Diazo160 | + | − | + | − | − | − | − |
| Diazo161 | − | − | + | − | − | − | − |
| Diazo162 | − | + | + | + | − | − | − |
| Diazo163 | − | + | + | + | − | − | − |
| Diazo164 | − | | + | − | − | − | − |
| Diazo165 | − | + | + | − | − | − | − |
| Diazo166 | − | − | + | − | − | − | − |
| Diazo167 | − | − | + | − | − | − | − |
| Diazo168 | + | − | + | + | − | − | − |
| Diazo169 | + | − | + | + | − | − | − |
| Diazo170 | + | − | + | + | − | − | − |
| Diazo171 | + | − | + | + | − | − | − |
| Diazo172 | + | − | + | + | − | − | − |
| Diazo173 | − | − | + | + | − | − | − |
| ACC174 | + | + | − | + | − | − | − |
| ACC175 | + | + | + | + | − | − | − |
| ACC176 | + | + | + | + | − | − | − |
| ACC177 | + | + | + | + | − | − | − |
| ACC178 | + | + | + | + | − | − | − |
| ACC179 | + | + | − | + | − | − | − |
| ACC180 | + | + | + | + | − | − | − |
| ACC181 | + | + | + | + | − | − | − |
| ACC182 | + | + | + | + | − | − | − |
| ACC183 | + | + | − | − | − | − | − |
| ACC184 | + | + | + | + | − | − | − |
| ACC185 | + | + | − | − | − | − | − |
| ACC186 | + | + | + | + | + | + | + |
| ACC187 | + | + | + | + | − | − | − |
| ACC188 | + | + | + | + | − | − | − |
| ACC189 | − | + | + | + | − | − | − |
| ACC190 | + | + | + | + | − | − | − |
| ACC191 | + | + | + | + | − | − | − |
| ACC192 | + | + | + | + | + | + | + |
| ACC193 | + | + | + | + | − | − | − |
| ACC194 | + | + | + | + | − | − | − |
| ACC195 | + | + | − | + | − | − | − |
| ACC196 | + | + | − | + | − | − | − |
| ACC197 | + | + | − | + | − | − | − |
| ACC198 | + | + | − | + | − | − | − |
| ACC199 | + | + | + | + | + | + | + |
| ACC200 | + | + | + | + | − | − | − |
| ACC201 | + | + | − | + | − | − | − |
| ACC202 | + | + | + | + | − | − | − |
| ACC203 | + | + | − | + | − | − | − |
| ACC204 | + | + | − | + | − | − | − |
| ACC205 | + | + | + | + | − | − | − |
| ACC206 | + | + | − | + | − | − | − |
| ACC207 | + | + | + | + | + | + | + |
| ACC208 | + | + | + | + | + | + | + |
| ACC209 | + | + | + | + | + | + | + |
| ACC210 | + | + | + | + | + | + | + |
| ACC211 | + | + | − | + | + | + | + |
| ACC212 | + | + | − | + | + | + | + |
| ACC213 | + | + | + | + | − | − | − |
| ACC214 | + | + | − | + | + | + | + |
| ACC215 | + | + | + | + | + | + | + |
| ACC216 | + | + | + | + | + | + | + |
| ACC217 | − | + | − | + | − | − | − |
| ACC218 | + | + | − | + | − | − | − |
| ACC219 | + | + | − | + | − | − | − |
| ACC220 | + | + | − | + | − | − | − |
| ACC221 | + | + | − | + | − | − | − |
| ACC222 | + | + | + | + | − | − | − |
| ACC223 | + | + | − | + | − | − | − |
| ACC224 | + | + | + | + | − | − | − |
| ACC225 | − | + | − | + | + | + | + |
| ACC226 | + | + | − | + | − | − | − |
| ACC227 | + | + | − | + | + | + | + |
| ACC228 | + | + | − | + | − | − | − |
| ACC229 | + | + | + | + | − | − | − |
| ACC230 | + | + | + | + | − | − | − |
| ACC231 | + | + | + | + | − | − | − |
| ACC232 | + | + | + | + | + | + | + |
| ACC233 | − | + | + | + | + | + | + |
| ACC234 | + | + | + | + | + | + | + |
| ACC235 | + | + | + | + | + | + | + |
| ACC236 | + | + | + | + | + | + | + |
| ACC237 | + | + | + | + | + | + | + |
| ACC238 | − | + | + | + | + | + | + |
| ACC239 | − | + | + | + | + | + | + |
| ACC240 | + | + | + | + | + | + | + |
| ACC241 | + | + | + | + | + | + | + |
| ACC242 | + | + | − | + | + | + | + |
| ACC243 | + | + | − | + | + | + | + |
| ACC244 | + | + | − | + | + | + | + |
| ACC245 | + | + | + | + | + | + | + |
| ACC246 | + | + | + | + | + | + | + |
| ACC247 | + | + | + | + | + | + | + |
| ACC248 | + | + | + | + | + | + | + |
| ACC249 | + | + | + | + | − | − | − |
| ACC250 | + | + | + | + | − | − | − |
| ACC251 | + | + | + | + | − | − | − |
| ACC252 | + | + | − | + | − | − | − |
| ACC253 | − | + | + | + | − | − | − |
| ACC254 | + | + | + | + | + | + | + |
| ACC255 | + | + | + | + | − | − | − |
| ACC256 | + | + | + | + | − | − | − |
| ACC257 | + | + | + | + | − | − | − |
| ACC258 | + | + | − | + | − | − | − |
| ACC259 | − | + | + | + | − | − | − |
| ACC260 | − | + | + | + | + | + | + |
| ACC261 | − | + | + | + | + | + | + |
| ACC262 | − | + | + | + | + | + | + |
| ACC263 | − | + | + | + | + | + | + |
| ACC264 | + | + | + | + | + | + | + |
| ACC265 | + | + | + | + | + | + | + |

TABLE 002
Bacterial microbiomes from site MGSAMP006,
multiple traits and heavy metal tolerance

| | Plant growth promotion traits | | | | | | |
|---|---|---|---|---|---|---|---|
| | | ACC | | | Heavy Metal | | |
| MG Isolate | Abscisic Acid | deaminase | Nitrogen | Phosphate | tolerance (2 mM) | | |
| ID | Degradation | activity | fixation | solubilisation | Cd | Zn | Pb |
| ABA272 | + | − | + | − | − | − | − |
| ABA273 | + | + | + | − | + | + | + |
| ABA274 | + | + | − | − | − | − | − |
| ABA275 | + | − | − | − | − | − | − |
| ABA276 | + | − | − | − | − | − | − |
| ABA277 | + | − | + | − | − | − | − |
| ABA278 | + | + | + | − | − | − | − |
| ABA279 | + | + | + | − | − | − | − |
| ABA280 | + | + | + | − | − | − | − |
| ABA281 | + | + | + | − | + | + | + |
| ABA282 | + | + | + | − | − | − | − |
| ABA283 | + | + | + | − | − | − | − |
| ABA284 | + | − | + | − | − | − | − |
| ABA285 | + | + | + | − | − | − | − |
| ABA286 | + | + | + | − | − | − | − |
| ABA287 | + | + | + | − | − | − | − |
| ABA288 | + | − | + | − | − | − | − |
| ABA289 | + | + | + | − | + | + | + |
| ABA290 | + | + | + | − | − | − | − |
| ABA291 | + | − | + | − | − | − | − |
| ABA292 | + | + | + | − | − | − | − |
| ABA293 | + | + | + | − | − | − | − |
| ABA294 | + | + | + | − | − | − | − |
| ABA295 | + | + | + | − | − | − | − |
| ABA296 | + | + | + | − | − | − | − |
| ABA297 | + | + | + | − | + | + | + |
| ABA298 | + | + | + | − | − | − | − |
| ABA299 | + | + | + | − | − | − | − |
| ABA300 | + | + | + | − | − | − | − |
| ABA301 | + | + | + | − | − | − | − |
| ABA302 | + | + | + | − | − | − | − |
| ABA303 | + | + | + | − | − | − | − |
| ABA304 | + | + | + | − | − | − | − |
| ABA305 | + | + | + | − | − | − | − |
| ABA306 | + | − | + | − | − | − | − |
| ABA307 | + | − | + | − | − | − | − |
| ABA308 | + | + | + | − | − | − | − |
| ABA309 | + | − | + | − | − | − | − |
| ABA310 | + | − | + | − | − | − | − |
| ABA311 | + | + | + | − | − | − | − |
| ABA312 | + | − | + | − | − | − | − |
| ABA313 | + | − | + | − | − | − | − |
| ABA314 | + | − | + | − | − | − | − |
| ABA315 | + | − | + | − | − | − | − |
| ABA316 | + | − | + | − | − | − | − |
| ABA317 | + | − | + | − | − | − | − |
| ABA318 | + | − | + | − | − | − | − |
| ABA319 | + | − | + | − | − | − | − |
| ABA320 | + | − | + | − | + | + | + |
| ABA321 | + | − | + | − | + | + | + |
| ABA322 | + | − | + | − | − | − | − |
| ABA323 | + | − | + | − | − | − | − |
| ABA324 | + | − | + | − | − | − | − |
| ABA325 | + | − | + | − | − | − | − |
| ABA326 | + | − | + | − | − | − | − |
| ABA327 | + | + | + | − | − | − | − |
| ABA328 | + | + | + | − | + | + | + |
| ABA329 | + | − | + | − | + | + | + |
| ABA330 | + | − | + | − | + | + | + |
| ABA331 | + | − | + | − | − | − | − |
| ABA332 | + | − | + | − | − | − | − |
| ABA333 | + | − | + | − | − | − | − |
| ABA334 | + | − | + | − | − | − | − |
| ABA335 | + | + | + | − | − | − | − |
| ABA336 | + | − | + | − | − | − | − |
| ABA337 | + | − | + | − | − | − | − |
| ABA338 | + | − | + | − | − | − | − |
| ABA339 | + | − | + | − | − | − | − |
| ABA340 | + | − | + | − | − | − | − |
| ABA341 | + | − | + | − | − | − | − |
| ABA342 | + | − | + | − | − | − | − |
| ABA343 | + | − | + | − | − | − | − |
| ABA344 | + | − | + | − | − | − | − |
| ABA345 | + | − | + | − | − | − | − |
| ABA346 | + | − | + | − | − | − | − |
| ABA347 | + | − | + | − | − | − | − |
| ABA348 | + | − | + | − | − | − | − |
| ABA349 | + | − | + | − | − | − | − |
| ABA350 | + | + | + | − | − | − | − |
| ABA351 | + | − | + | − | − | − | − |
| ABA352 | + | − | − | − | − | − | − |
| ABA353 | + | − | + | − | − | − | − |
| ABA354 | + | − | + | − | − | − | − |
| ABA355 | + | + | + | − | − | − | − |
| ABA356 | + | − | + | − | − | − | − |
| ABA357 | + | + | + | − | − | − | − |
| ABA358 | + | − | + | − | − | − | − |
| ABA359 | + | + | + | − | − | − | − |
| ABA360 | + | + | − | − | − | − | − |
| ABA361 | + | + | + | − | − | − | − |
| ABA362 | + | + | − | − | − | − | − |
| ABA363 | + | + | + | − | | | |
| CZL364 | − | − | − | + | + | + | + |
| CZL365 | − | + | − | + | + | + | + |
| CZL366 | − | + | + | − | + | + | + |
| CZL367 | − | − | − | − | + | + | + |
| CZL368 | − | − | − | + | + | + | + |
| CZL369 | − | − | − | + | + | + | + |
| CZL370 | − | − | − | − | + | + | + |
| CZL371 | − | − | + | − | + | + | + |
| CZL372 | − | + | + | + | + | + | + |
| CZL373 | − | + | + | + | + | + | + |
| CZL374 | − | + | − | − | + | + | + |
| CZL375 | − | − | − | − | + | + | + |
| CZL376 | − | − | − | − | + | + | + |
| CZL377 | − | − | − | − | + | + | + |
| CZL378 | − | − | − | + | + | + | + |
| CZL379 | − | + | − | + | + | + | + |
| CZL380 | − | + | + | + | + | + | + |
| CZL381 | − | + | + | + | + | + | + |
| CZL382 | − | + | − | − | + | + | + |
| CZL383 | − | − | − | − | + | + | + |
| CZL384 | − | − | − | − | + | + | + |
| CZL385 | − | − | − | − | + | + | + |
| CZL386 | − | + | − | + | + | + | + |
| CZL387 | − | + | − | + | + | + | + |
| CZL388 | − | + | + | − | + | + | + |
| CZL389 | − | + | + | + | + | + | + |
| CZL390 | − | + | − | − | + | + | + |
| CZL391 | − | − | − | − | + | + | + |
| CZL392 | − | − | − | − | + | + | + |
| CZL393 | − | − | − | − | + | + | + |
| CZL394 | − | + | − | − | + | + | + |
| CZL395 | − | + | − | − | + | + | + |
| CZL396 | − | + | + | − | + | + | + |
| CZL397 | − | + | − | − | + | + | + |
| CZL398 | − | + | − | − | + | + | + |
| CZL399 | − | + | − | − | + | + | + |
| CZL400 | − | + | − | − | + | + | + |
| CZL401 | − | − | − | + | + | + | + |
| CZL402 | − | + | − | − | + | + | + |
| CZL403 | − | − | + | + | + | + | + |
| CZL404 | − | + | − | − | + | + | + |
| CZL405 | − | − | + | − | + | + | + |
| CZL406 | − | + | + | − | + | + | + |
| CZL407 | − | + | − | − | + | + | + |
| CZL408 | − | + | − | − | + | + | + |
| CZL409 | − | + | − | + | + | + | + |
| CZL410 | − | − | − | + | + | + | + |
| CZL411 | − | − | − | − | + | + | + |
| CZL412 | − | − | − | − | + | + | + |
| Diazo413 | − | + | + | + | + | + | + |
| Diazo414 | − | + | + | − | − | − | − |
| Diazo415 | − | + | + | − | + | + | + |
| Diazo416 | − | − | + | + | − | − | − |
| Diazo417 | − | + | + | − | − | − | − |
| Diazo418 | − | + | + | − | + | + | + |
| Diazo419 | − | − | + | − | − | − | − |
| Diazo420 | − | + | + | − | + | + | + |
| Diazo421 | − | − | + | − | − | − | − |
| Diazo422 | − | + | + | − | + | + | + |
| Diazo423 | − | + | + | + | + | + | + |
| Diazo424 | − | − | + | + | − | − | − |
| Diazo425 | − | − | + | + | − | − | − |
| Diazo426 | − | − | + | − | − | − | − |
| Diazo427 | − | + | + | − | − | − | − |
| Diazo428 | − | + | + | − | − | − | − |
| Diazo429 | − | − | + | − | − | − | − |
| Diazo430 | − | + | + | − | + | + | + |
| Diazo431 | − | − | + | + | − | − | − |
| Diazo432 | − | + | + | − | − | − | − |
| Diazo433 | − | − | + | − | − | − | − |
| Diazo434 | − | − | + | − | − | − | − |
| Diazo435 | − | + | + | − | − | − | − |
| Diazo436 | − | + | + | − | − | − | − |
| Diazo437 | − | + | + | − | + | + | + |
| Diazo438 | − | − | + | − | − | − | − |
| Diazo439 | − | − | + | − | − | − | − |
| Diazo440 | − | − | + | − | − | − | − |
| Diazo441 | − | − | + | − | − | − | − |
| Diazo442 | − | − | + | − | − | − | − |
| Diazo443 | − | − | + | + | + | + | + |
| Diazo444 | − | − | + | + | + | + | + |
| Diazo445 | − | + | + | − | − | − | − |
| Diazo446 | − | + | + | − | + | + | + |
| Diazo447 | − | + | + | + | + | + | + |
| Diazo448 | − | + | + | + | + | + | + |
| Diazo449 | − | + | + | + | + | + | + |
| Diazo450 | − | − | + | − | − | − | − |
| Diazo451 | − | + | + | + | − | − | − |
| Diazo452 | − | + | + | + | − | − | − |
| Diazo453 | − | + | + | − | − | − | − |
| Diazo454 | − | − | + | − | + | + | + |
| Diazo455 | − | + | + | + | + | + | + |
| Diazo456 | − | + | + | + | + | + | + |
| Diazo457 | − | − | + | + | + | + | + |
| Diazo458 | − | + | + | − | − | − | − |
| Diazo459 | − | − | + | − | − | − | − |
| Diazo460 | − | + | + | − | + | + | + |
| Diazo461 | − | + | + | − | + | + | + |
| Diazo462 | − | − | + | − | | | |
| Diazo463 | − | + | + | + | + | + | + |
| Diazo464 | − | + | + | + | + | + | + |
| Diazo465 | − | + | + | + | + | + | + |
| Diazo466 | − | + | + | + | − | − | − |
| Diazo467 | − | − | + | − | − | − | − |
| Diazo468 | − | − | + | − | − | − | − |
| PO4_469 | − | + | + | + | + | + | + |
| PO4_470 | − | + | + | + | − | − | − |
| PO4_471 | − | + | + | + | + | + | + |
| PO4_472 | − | + | + | + | + | + | + |
| PO4_473 | − | + | + | + | + | + | + |
| PO4_474 | − | + | + | + | + | + | + |
| PO4_475 | − | + | + | + | + | + | + |
| PO4_476 | − | + | + | + | + | + | + |
| PO4_477 | − | + | + | + | + | + | + |
| PO4_478 | − | + | + | + | + | + | + |
| PO4_479 | − | + | + | + | + | + | + |
| PO4_480 | − | + | + | + | + | + | + |
| PO4_481 | − | + | + | + | + | + | + |
| PO4_482 | − | + | + | + | + | + | + |
| PO4_483 | − | + | + | + | + | + | + |
| PO4_484 | − | + | + | + | + | + | + |
| PO4_485 | − | + | + | + | + | + | + |
| PO4_486 | − | + | + | + | + | + | + |
| PO4_487 | − | + | + | + | + | + | + |
| PO4_488 | − | + | + | + | + | + | + |
| PO4_489 | − | + | + | + | + | + | + |
| PO4_490 | − | + | + | + | + | + | + |
| PO4_491 | − | + | + | + | + | + | + |
| PO4_492 | − | + | + | + | + | + | + |
| PO4_493 | − | + | + | + | − | − | − |
| PO4_494 | − | + | + | + | + | + | + |
| PO4_495 | − | + | + | + | + | + | + |
| PO4_496 | − | + | + | + | + | + | + |
| PO4_497 | − | + | + | + | + | + | + |
| PO4_498 | − | + | + | + | + | + | + |
| PO4_499 | − | + | + | + | + | + | + |
| PO4_500 | − | + | + | + | + | + | + |
| PO4_501 | − | + | + | + | + | + | + |
| PO4_502 | − | + | + | + | − | − | − |
| PO4_503 | − | + | + | + | + | + | + |
| PO4_504 | − | + | + | + | + | + | + |
| PO4_505 | − | + | + | + | + | + | + |
| PO4_506 | − | + | + | + | + | + | + |
| PO4_507 | − | + | + | + | + | + | + |
| PO4_508 | − | + | + | + | + | + | + |
| PO4_509 | − | + | + | + | − | − | − |
| PO4_510 | − | − | + | + | − | − | − |
| PO4_511 | − | + | + | + | + | + | + |
| PO4_512 | − | + | + | + | + | + | + |
| PO4_513 | − | + | + | + | + | + | + |
| PO4_514 | − | + | + | + | + | + | + |
| PO4_515 | − | + | + | + | + | + | + |
| PO4_516 | − | + | + | + | + | + | + |
| PO4_517 | − | + | + | + | + | + | + |
| PO4_518 | − | + | + | + | + | + | + |
| PO4_519 | − | + | + | + | + | + | + |
| PO4_520 | − | + | + | + | + | + | + |
| PO4_521 | − | − | + | + | − | − | − |
| PO4_522 | − | + | + | + | + | + | + |
| PO4_523 | − | + | + | + | | | |
| PO4_524 | − | + | + | + | + | + | + |
| PO4_525 | − | + | + | + | − | − | − |
| PO4_526 | − | − | + | + | − | − | − |
| PO4_527 | − | + | + | + | + | + | + |
| PO4_528 | − | + | + | + | + | + | + |
| PO4_529 | − | − | + | + | − | − | − |
| PO4_530 | − | + | + | + | − | − | − |
| PO4_531 | − | + | + | + | − | − | − |
| PO4_532 | − | + | + | + | − | − | − |
| PO4_533 | − | + | + | + | − | − | − |
| PO4_534 | − | + | − | + | − | − | − |
| PO4_535 | − | + | + | + | − | − | − |
| PO4_536 | − | + | + | + | − | − | − |
| PO4_537 | − | − | + | + | − | − | − |
| PO4_538 | − | + | + | + | + | + | + |
| PO4_539 | − | + | + | + | + | + | + |
| PO4_540 | − | + | + | + | + | + | + |
| PO4_541 | − | + | + | + | − | − | − |
| PO4_542 | − | + | + | + | − | − | − |
| PO4_543 | − | + | + | + | − | − | − |
| PO4_544 | − | + | + | + | + | + | + |
| PO4_545 | − | + | + | + | − | − | − |
| PO4_546 | − | + | + | + | − | − | − |
| PO4_547 | − | − | + | + | + | + | + |
| PO4_548 | − | + | + | + | + | + | + |
| PO4_549 | − | + | + | + | − | − | − |
| PO4_550 | − | + | + | + | − | − | − |
| PO4_551 | − | + | + | + | − | − | − |
| PO4_552 | − | + | + | + | + | + | + |
| PO4_553 | − | + | + | + | + | + | + |
| PO4_554 | − | + | + | + | + | + | + |
| PO4_555 | − | + | + | + | + | + | + |
| PO4_556 | − | + | + | + | + | + | + |
| PO4_557 | − | + | + | + | − | − | − |
| PO4_558 | − | + | + | + | + | + | + |
| PO4_559 | − | + | + | + | + | + | + |
| PO4_560 | − | + | + | + | + | + | + |
| ACC561 | − | + | + | − | − | − | − |
| ACC562 | − | + | − | + | − | − | − |
| ACC563 | − | + | + | − | + | + | + |
| ACC564 | − | + | + | + | − | − | − |
| ACC565 | − | + | − | + | − | − | − |
| ACC566 | − | + | − | − | − | − | − |
| ACC567 | − | + | + | − | − | − | − |
| ACC568 | − | + | + | − | − | − | − |
| ACC569 | − | + | + | − | + | + | + |
| ACC570 | − | + | + | + | − | − | − |
| ACC571 | − | + | + | − | − | − | − |
| ACC572 | − | + | + | − | − | − | − |
| ACC573 | − | + | + | − | − | − | − |
| ACC574 | − | + | + | − | − | − | − |
| ACC575 | − | + | + | − | − | − | − |
| ACC576 | − | + | + | − | − | − | − |
| ACC577 | − | + | + | − | + | + | + |
| ACC578 | − | + | + | + | + | + | + |
| ACC579 | − | + | + | − | + | + | + |
| ACC580 | − | + | + | + | − | − | − |
| ACC581 | − | + | + | − | − | − | − |
| ACC582 | − | + | + | − | − | − | − |
| ACC583 | − | + | + | − | − | − | − |
| ACC584 | − | + | + | − | − | − | − |
| ACC585 | − | + | + | − | − | − | − |
| ACC586 | − | + | + | − | − | − | − |
| ACC587 | − | + | + | − | − | − | − |
| ACC588 | − | + | + | − | − | − | − |
| ACC589 | − | + | + | + | − | − | − |
| ACC590 | − | + | + | − | − | − | − |
| ACC591 | − | + | + | − | − | − | − |
| ACC592 | − | + | + | − | − | − | − |
| ACC593 | − | + | + | − | − | − | − |
| ACC594 | − | + | + | − | − | − | − |
| ACC595 | − | + | + | − | − | − | − |
| ACC596 | − | + | − | + | − | − | − |
| ACC597 | − | + | − | + | − | − | − |
| ACC598 | − | + | + | − | − | − | − |
| ACC599 | − | + | + | − | − | − | − |
| ACC600 | − | + | + | − | − | − | − |
| ACC601 | − | + | + | − | − | − | − |
| ACC602 | − | + | + | − | − | − | − |
| ACC603 | − | + | + | − | − | − | − |
| ACC604 | − | + | − | + | − | − | − |
| ACC605 | − | + | − | + | − | − | − |
| ACC606 | − | + | + | − | − | − | − |
| ACC607 | − | + | + | − | − | − | − |
| ACC608 | − | + | + | − | − | − | − |
| ACC609 | − | + | − | + | − | − | − |
| ACC610 | − | + | − | − | − | − | − |
| ACC611 | − | + | − | + | − | − | − |
| ACC612 | − | + | + | − | − | − | − |
| ACC613 | − | + | + | − | − | − | − |
| ACC614 | − | + | + | − | − | − | − |
| ACC615 | − | + | + | − | − | − | − |
| ACC616 | − | + | + | − | − | − | − |
| ACC617 | − | + | − | + | − | − | − |
| ACC618 | − | + | + | − | − | − | − |
| ACC619 | − | + | + | − | − | − | − |
| ACC620 | − | + | + | − | − | − | − |
| ACC621 | − | + | + | − | − | − | − |
| ACC622 | − | + | + | − | − | − | − |
| ACC623 | − | + | + | − | − | − | − |
| ACC624 | − | + | + | − | − | − | − |
| ACC625 | − | + | + | − | − | − | − |
| ACC626 | − | + | + | − | − | − | − |
| ACC627 | − | + | + | − | − | − | − |
| ACC628 | − | + | + | − | − | − | − |
| ACC629 | − | + | + | − | − | − | − |
| ACC630 | − | + | + | − | − | − | − |
| ACC631 | − | + | + | − | − | − | − |
| ACC632 | − | + | + | − | − | − | − |
| ACC633 | − | + | + | − | − | − | − |
| ACC634 | − | + | + | − | − | − | − |
| ACC635 | − | + | + | − | − | − | − |
| ACC636 | − | + | + | − | − | − | − |
| ACC637 | − | + | + | − | − | − | − |
| ACC638 | − | + | + | − | − | − | − |
| ACC639 | − | + | + | − | − | − | − |
| ACC640 | − | + | + | − | − | − | − |
| ACC641 | − | + | + | − | − | − | − |
| ACC642 | − | + | + | − | − | − | − |
| ACC643 | − | + | + | − | − | − | − |
| ACC644 | − | + | + | − | − | − | − |
| ACC645 | − | + | + | − | − | − | − |
| ACC646 | − | + | + | − | − | − | − |
| ACC647 | − | + | + | − | − | − | − |
| ACC648 | − | + | + | − | − | − | − |
| ACC649 | − | + | + | − | − | − | − |
| ACC650 | − | + | + | − | − | − | − |
| ACC651 | − | + | + | − | − | − | − |
| ACC652 | − | + | + | − | − | − | − |

TABLE 3
Bacterial microbiomes from site MGSAMP008,
multiple traits and heavy metal tolerance

| | Plant growth promotion traits | | | | | | |
|---|---|---|---|---|---|---|---|
| | | ACC | | | Heavy Metal | | |
| MG Isolate | Abscisic Acid | deaminase | Nitrogen | Phosphate | tolerance (2 mM) | | |
| ID | Degradation | activity | fixation | solubilisation | Cd | Zn | Pb |
| ABA653 | + | − | + | + | − | − | − |
| ABA654 | + | − | + | + | − | − | − |
| ABA655 | + | − | + | − | − | − | − |
| ABA656 | + | − | + | − | + | + | + |
| ABA657 | + | − | + | − | − | − | − |
| ABA658 | + | − | + | − | − | − | − |
| ABA659 | + | + | + | − | − | − | − |
| ABA660 | + | + | + | − | + | + | + |
| ABA661 | + | + | + | + | + | + | + |
| ABA662 | + | + | + | + | + | + | + |
| ABA663 | + | + | + | − | + | + | + |
| ABA664 | + | + | + | − | + | + | + |
| ABA665 | + | + | + | − | − | − | − |
| ABA666 | + | + | + | − | − | − | − |
| ABA667 | + | + | + | − | − | − | − |
| ABA668 | + | + | + | − | − | − | − |
| ABA669 | + | − | + | + | + | + | + |
| ABA670 | + | − | + | + | − | − | − |
| ABA671 | + | − | + | − | − | − | − |
| ABA672 | + | − | + | − | + | + | + |
| ABA673 | + | − | + | − | − | − | − |
| ABA674 | + | − | + | + | − | − | − |
| ABA675 | + | + | + | − | − | − | − |
| ABA676 | + | + | + | − | − | − | − |
| ABA677 | + | − | + | − | − | − | − |
| ABA678 | + | − | + | − | − | − | − |
| ABA679 | + | − | + | − | − | − | − |
| ABA680 | + | − | + | − | + | + | + |
| ABA681 | + | − | + | + | + | + | + |
| ABA682 | + | − | + | − | − | − | − |
| ABA683 | + | + | + | + | + | + | + |
| ABA684 | + | + | + | − | − | − | − |
| ABA685 | + | − | + | − | − | − | − |
| ABA686 | + | − | + | − | − | − | − |
| ABA687 | + | − | + | − | − | − | − |
| ABA688 | + | − | + | − | − | − | − |
| ABA689 | + | − | + | + | + | + | + |
| ABA690 | + | − | + | + | + | + | + |
| ABA691 | + | + | + | − | − | − | − |
| ABA692 | + | + | + | − | − | − | − |
| ABA693 | + | − | − | − | − | − | − |
| ABA694 | + | − | − | − | − | − | − |
| ABA695 | + | − | + | − | − | − | − |
| ABA696 | + | − | + | − | − | − | − |
| ABA697 | + | − | + | + | + | + | + |
| ABA698 | + | − | + | + | + | + | + |
| ABA699 | + | + | + | − | + | + | + |
| ABA700 | + | + | − | − | − | − | − |
| ABA701 | + | − | − | − | − | − | − |
| ABA702 | + | − | − | − | − | − | − |
| ABA703 | + | − | + | − | − | − | − |
| ABA704 | + | − | + | − | − | − | − |
| ABA705 | + | − | + | + | + | + | + |
| ABA706 | + | − | + | + | + | + | + |
| ABA707 | + | + | − | + | − | − | − |
| ABA708 | + | + | + | − | − | − | − |
| ABA709 | + | + | + | − | − | − | − |
| ABA710 | + | − | − | − | − | − | − |
| ABA711 | + | − | + | − | − | − | − |
| ABA712 | + | − | + | − | − | − | − |
| ABA713 | + | − | + | − | − | − | − |
| ABA714 | + | − | + | − | − | − | − |
| ABA715 | + | + | + | − | − | − | − |
| ABA716 | + | + | + | − | − | − | − |
| ABA717 | + | − | + | − | − | − | − |
| ABA718 | + | − | + | − | − | − | − |
| ABA719 | + | − | + | − | − | − | − |
| ABA720 | + | − | + | − | − | − | − |
| ABA721 | + | − | + | − | + | + | + |
| ABA722 | + | − | + | − | − | − | − |
| ABA723 | + | + | + | − | − | − | − |
| ABA724 | + | + | + | + | − | − | − |
| ABA725 | + | − | + | − | − | − | − |
| ABA726 | + | − | + | − | − | − | − |
| ABA727 | + | − | + | − | − | − | − |
| ABA728 | + | − | + | − | − | − | − |
| ABA729 | + | + | + | − | − | − | − |
| ABA730 | + | + | + | − | − | − | − |
| ABA731 | + | + | + | + | − | − | − |
| ABA732 | + | + | + | + | − | − | − |
| ABA733 | + | − | + | − | − | − | − |
| ABA734 | + | − | + | − | − | − | − |
| ABA735 | + | − | + | − | − | − | − |
| ABA736 | + | − | + | − | − | − | − |
| ABA737 | + | + | + | − | − | − | − |
| ABA738 | + | + | + | − | − | − | − |
| ABA739 | + | + | + | − | − | − | − |
| ABA740 | + | + | + | − | − | − | − |
| ABA741 | + | + | + | + | − | − | − |
| ABA742 | + | + | + | − | − | − | − |
| ABA743 | + | + | + | − | − | − | − |
| ABA744 | + | + | + | − | − | − | − |
| CZL745 | + | + | + | + | + | + | + |
| CZL746 | + | + | + | + | + | + | + |
| CZL747 | + | + | + | + | + | + | + |
| CZL748 | + | + | + | + | + | + | + |
| CZL749 | + | + | + | + | + | + | + |
| CZL750 | + | + | + | + | + | + | + |
| CZL751 | + | + | + | + | + | + | + |
| CZL752 | + | + | + | + | + | + | + |
| CZL753 | + | + | + | + | + | + | + |
| CZL754 | + | + | + | + | + | + | + |
| CZL755 | + | + | + | + | + | + | + |
| CZL756 | + | + | + | + | + | + | + |
| CZL757 | + | + | + | + | + | + | + |
| CZL758 | + | + | + | + | + | + | + |
| CZL759 | + | + | + | + | + | + | + |
| CZL760 | + | + | + | + | + | + | + |
| CZL761 | + | + | + | + | + | + | + |
| CZL762 | + | + | + | + | + | + | + |
| CZL763 | + | + | + | + | + | + | + |
| CZL764 | + | + | + | + | + | + | + |
| CZL765 | + | + | + | + | + | + | + |
| CZL766 | + | + | + | + | + | + | + |
| CZL767 | + | + | + | + | + | + | + |
| CZL768 | + | + | + | + | + | + | + |
| CZL769 | + | + | + | + | + | + | + |
| CZL770 | + | + | + | + | + | + | + |
| CZL771 | + | + | + | + | + | + | + |
| CZL772 | + | + | + | + | + | + | + |
| CZL773 | + | + | + | + | + | + | + |
| CZL774 | + | + | + | + | + | + | + |
| CZL775 | + | + | + | + | + | + | + |
| CZL776 | + | + | + | + | + | + | + |
| CZL777 | + | + | + | + | + | + | + |
| CZL778 | + | + | + | + | + | + | + |
| CZL779 | + | + | + | + | + | + | + |
| CZL780 | + | + | + | + | + | + | + |
| CZL781 | + | + | + | + | + | + | + |
| CZL782 | + | + | + | + | + | + | + |
| CZL783 | + | + | + | + | + | + | + |
| CZL784 | + | + | + | + | + | + | + |
| CZL785 | + | + | + | + | + | + | + |
| CZL786 | + | + | + | + | + | + | + |
| CZL787 | + | + | + | + | + | + | + |
| CZL788 | + | + | + | + | + | + | + |
| CZL789 | + | + | + | + | + | + | + |
| CZL790 | + | + | + | + | + | + | + |
| CZL791 | + | + | + | + | + | + | + |
| CZL792 | + | + | + | + | + | + | + |
| CZL793 | + | + | + | + | + | + | + |
| CZL794 | + | + | + | + | + | + | + |
| CZL795 | + | + | + | + | + | + | + |
| CZL796 | + | + | + | + | + | + | + |
| CZL797 | + | + | + | + | + | + | + |
| CZL798 | + | + | + | + | + | + | + |
| CZL799 | + | + | + | + | + | + | + |
| CZL800 | + | + | + | + | + | + | + |
| CZL801 | + | + | + | + | + | + | + |
| CZL802 | + | + | + | + | + | + | + |
| CZL803 | + | + | + | + | + | + | + |
| CZL804 | + | + | + | + | + | + | + |
| CZL805 | + | + | + | + | + | + | + |
| CZL806 | + | + | + | + | + | + | + |
| CZL807 | + | + | + | + | + | + | + |
| CZL808 | + | + | + | + | + | + | + |
| CZL809 | + | + | + | + | + | + | + |
| CZL810 | + | + | + | + | + | + | + |
| CZL811 | + | + | + | + | + | + | + |
| CZL812 | + | + | + | + | + | + | + |
| CZL813 | + | + | + | + | + | + | + |
| CZL814 | + | + | + | + | + | + | + |
| CZL815 | + | + | + | + | + | + | + |
| CZL816 | + | + | + | + | + | + | + |
| CZL817 | + | + | + | + | + | + | + |
| CZL818 | + | + | + | + | + | + | + |
| CZL819 | + | + | + | + | + | + | + |
| CZL820 | + | + | + | + | + | + | + |
| CZL821 | + | + | + | + | + | + | + |
| CZL822 | + | + | + | + | + | + | + |
| CZL823 | + | + | + | + | + | + | + |
| CZL824 | + | + | + | + | + | + | + |
| CZL825 | + | + | + | + | + | + | + |
| CZL826 | + | + | + | + | + | + | + |
| CZL827 | + | + | + | + | + | + | + |
| CZL828 | + | + | + | + | + | + | + |
| CZL829 | + | + | + | + | + | + | + |
| CZL830 | + | + | + | + | + | + | + |
| CZL831 | + | + | + | + | + | + | + |
| CZL832 | + | + | + | + | + | + | + |
| CZL833 | + | + | + | + | + | + | + |
| CZL834 | − | − | + | + | + | + | + |
| CZL835 | − | − | + | + | + | + | + |
| CZL836 | + | + | + | + | + | + | + |
| Diazo837 | − | + | + | + | − | − | − |
| Diazo838 | − | + | + | + | − | − | − |
| Diazo839 | − | + | + | + | − | − | − |
| Diazo840 | − | + | + | + | − | − | − |
| Diazo841 | − | + | + | + | − | − | − |
| Diazo842 | − | + | + | + | − | − | − |
| Diazo843 | − | + | + | + | − | − | − |
| Diazo844 | − | + | + | + | + | + | + |
| Diazo845 | − | + | + | + | + | + | + |
| Diazo846 | − | + | + | + | + | + | + |
| Diazo847 | − | + | + | + | − | − | − |
| Diazo848 | − | + | + | + | − | − | − |
| Diazo849 | − | − | + | + | − | − | − |
| Diazo850 | − | + | + | + | − | − | − |
| Diazo851 | − | + | + | + | − | − | − |
| Diazo852 | − | + | + | + | + | + | + |
| Diazo853 | − | + | + | + | − | − | − |
| Diazo854 | − | + | + | + | − | − | − |
| Diazo855 | − | + | + | + | − | − | − |
| Diazo856 | − | + | + | + | − | − | − |
| Diazo857 | − | − | + | + | − | − | − |
| Diazo858 | − | + | + | + | + | + | + |
| Diazo859 | − | + | + | + | − | − | − |
| Diazo860 | − | + | + | + | − | − | − |
| Diazo861 | − | + | + | + | + | + | + |
| Diazo862 | − | + | + | + | − | − | − |
| Diazo863 | − | + | + | + | − | − | − |
| Diazo864 | − | + | + | + | + | + | + |
| Diazo865 | − | + | + | + | + | + | + |
| Diazo866 | − | + | + | + | − | − | − |
| Diazo867 | − | + | + | + | − | − | − |
| Diazo868 | − | + | + | + | − | − | − |
| Diazo869 | − | + | + | + | − | − | − |
| Diazo870 | − | + | + | − | − | − | − |
| Diazo871 | − | + | + | − | − | − | − |
| Diazo872 | − | + | + | + | − | − | − |
| Diazo873 | − | + | + | + | + | + | + |
| Diazo874 | − | − | + | − | − | − | − |
| Diazo875 | − | − | + | − | − | − | − |
| Diazo876 | − | + | + | + | − | − | − |
| Diazo877 | − | + | + | + | − | − | − |
| Diazo878 | − | − | + | − | − | − | − |
| Diazo879 | − | + | + | + | − | − | − |
| Diazo880 | − | + | + | + | − | − | − |
| Diazo881 | − | − | + | + | − | − | − |
| Diazo882 | − | + | + | − | − | − | − |
| Diazo883 | − | + | + | − | − | − | − |
| Diazo884 | − | + | + | + | − | − | − |
| Diazo885 | − | + | + | + | − | − | − |
| Diazo886 | − | + | + | + | − | − | − |
| Diazo887 | − | + | + | + | − | − | − |
| Diazo888 | − | + | + | + | − | − | − |
| Diazo889 | − | − | + | + | − | − | − |
| Diazo890 | − | − | + | − | − | − | − |
| Diazo891 | − | + | + | + | − | − | − |
| Diazo892 | − | + | + | + | − | − | − |
| Diazo893 | − | + | + | + | − | − | − |
| Diazo894 | − | + | + | + | − | − | − |
| Diazo895 | − | + | + | + | − | − | − |
| Diazo896 | − | + | + | + | − | − | − |
| Diazo897 | − | − | + | + | − | − | − |
| Diazo898 | − | − | + | − | − | − | − |
| Diazo899 | − | + | + | + | − | − | − |
| Diazo900 | − | + | + | + | + | + | + |
| Diazo901 | − | + | + | + | − | − | − |
| Diazo902 | − | + | + | + | − | − | − |
| Diazo903 | − | + | + | + | − | − | − |
| Diazo904 | − | + | + | + | − | − | − |
| Diazo905 | − | − | + | + | − | − | − |
| Diazo906 | − | − | + | − | − | − | − |
| Diazo907 | − | + | + | − | − | − | − |
| Diazo908 | − | + | + | + | + | + | + |
| Diazo909 | − | + | + | + | + | + | + |
| Diazo910 | − | + | + | − | − | − | − |
| Diazo911 | − | + | + | − | − | − | − |
| Diazo912 | − | + | + | − | − | − | − |
| Diazo913 | − | + | + | − | − | − | − |
| Diazo914 | − | − | + | − | − | − | − |
| Diazo915 | − | − | + | − | − | − | − |
| Diazo916 | − | + | + | + | + | + | + |
| Diazo917 | − | + | + | + | + | + | + |
| Diazo918 | − | + | + | − | − | − | − |
| Diazo919 | − | − | + | − | − | − | − |
| Diazo920 | − | − | + | − | − | − | − |
| Diazo921 | − | + | + | − | − | − | − |
| Diazo922 | − | − | + | − | − | − | − |
| Diazo923 | − | + | + | − | − | − | − |
| Diazo924 | − | + | + | − | + | + | + |
| Diazo925 | − | − | + | − | − | − | − |
| Diazo926 | − | − | + | − | − | − | − |
| Diazo927 | − | + | + | − | − | − | − |
| Diazo928 | − | − | + | − | − | − | − |
| PO4_929 | − | + | + | + | − | − | − |
| PO4_930 | − | − | + | + | + | + | + |
| PO4_931 | − | + | + | + | − | − | − |
| PO4_932 | − | − | + | + | + | + | + |
| PO4_933 | − | − | + | + | + | + | + |
| PO4_934 | − | − | + | + | + | + | + |
| PO4_935 | − | − | + | + | + | + | + |
| PO4_936 | − | − | + | + | + | + | + |
| PO4_937 | − | − | + | + | + | + | + |
| PO4_938 | − | − | + | + | + | + | + |
| PO4_939 | − | − | + | + | + | + | + |
| PO4_940 | − | − | + | + | + | + | + |
| PO4_941 | − | − | + | + | + | + | + |
| PO4_942 | − | − | + | + | + | + | + |
| PO4_943 | − | − | + | + | + | + | + |
| PO4_944 | − | − | + | + | + | + | + |
| PO4_945 | − | − | + | + | + | + | + |
| PO4_946 | − | − | + | + | + | + | + |
| PO4_947 | − | − | + | + | + | + | + |
| PO4_948 | − | − | + | + | + | + | + |
| PO4_949 | − | − | + | + | + | + | + |
| PO4_950 | − | − | + | + | + | + | + |
| PO4_951 | − | − | + | + | + | + | + |
| PO4_952 | − | − | + | + | + | + | + |
| PO4_953 | − | − | + | + | + | + | + |
| PO4_954 | − | − | + | + | − | − | − |
| PO4_955 | − | − | + | + | − | − | − |
| PO4_956 | − | − | + | + | + | + | + |
| PO4_957 | − | − | + | + | + | + | + |
| PO4_958 | − | − | + | + | + | + | + |
| PO4_959 | − | − | + | + | + | + | + |
| PO4_960 | − | − | + | + | + | + | + |
| PO4_961 | − | + | | + | − | − | − |
| PO4_962 | − | + | | + | − | − | − |
| PO4_963 | − | − | + | + | − | − | − |
| PO4_964 | − | − | + | + | − | − | − |
| PO4_965 | − | − | + | + | − | − | − |
| PO4_966 | − | − | + | + | + | + | + |
| PO4_967 | − | − | + | + | + | + | + |
| PO4_968 | − | − | + | + | + | + | + |
| PO4_969 | − | + | + | + | − | − | − |
| PO4_970 | − | + | + | + | − | − | − |
| PO4_971 | − | + | + | + | − | − | − |
| PO4_972 | − | + | + | + | − | − | − |
| PO4_973 | − | + | + | + | − | − | − |
| PO4_974 | − | + | + | + | − | − | − |
| PO4_975 | − | − | + | + | + | + | + |
| PO4_976 | − | − | + | + | + | + | + |
| PO4_977 | − | − | + | + | − | − | − |
| PO4_978 | − | + | + | + | − | − | − |
| PO4_979 | − | + | + | + | − | − | − |
| PO4_980 | − | + | + | + | − | − | − |
| PO4_981 | − | + | + | + | − | − | − |
| PO4_982 | − | − | + | + | − | − | − |
| PO4_983 | − | − | + | + | + | + | + |
| PO4_984 | − | − | + | + | + | + | + |
| PO4_985 | − | − | + | + | − | − | − |
| PO4_986 | − | + | + | + | − | − | − |
| PO4_987 | − | + | + | + | − | − | − |
| PO4_988 | − | + | + | + | − | − | − |
| PO4_989 | − | + | + | + | − | − | − |
| PO4_990 | − | − | + | + | − | − | − |
| PO4_991 | − | − | + | + | + | + | + |
| PO4_992 | − | − | + | + | + | + | + |
| PO4_993 | − | − | + | + | − | − | − |
| PO4_994 | − | + | + | + | − | − | − |
| PO4_995 | − | + | + | + | − | − | − |
| PO4_996 | − | + | + | + | − | − | − |
| PO4_997 | − | + | + | + | − | − | − |
| PO4_998 | − | − | + | + | − | − | − |
| PO4_999 | − | − | + | + | + | + | + |
| PO4_1000 | − | − | + | + | + | + | + |
| PO4_1001 | − | − | + | + | − | − | − |
| PO4_1002 | − | + | + | + | − | − | − |
| PO4_1003 | − | + | + | + | − | − | − |
| PO4_1004 | − | + | + | + | − | − | − |
| PO4_1005 | − | + | + | + | − | − | − |
| PO4_1006 | − | + | + | + | − | − | − |
| PO4_1007 | − | − | + | + | + | + | + |
| PO4_1008 | − | − | + | + | − | − | − |
| PO4_1009 | − | − | + | + | − | − | − |
| PO4_1010 | − | + | + | + | − | − | − |
| PO4_1011 | − | + | + | + | − | − | − |
| PO4_1012 | − | + | + | + | − | − | − |
| PO4_1013 | − | + | + | + | − | − | − |
| PO4_1014 | − | + | + | + | − | − | − |
| PO4_1015 | − | + | + | + | − | − | − |
| PO4_1016 | − | − | + | + | − | − | − |
| PO4_1017 | − | − | + | + | − | − | − |
| PO4_1018 | − | − | + | + | − | − | − |
| PO4_1019 | − | − | + | + | − | − | − |
| PO4_1020 | − | − | + | + | − | − | − |
| ACC1021 | − | + | + | − | + | + | + |
| ACC1022 | − | + | + | − | − | − | − |
| ACC1023 | − | + | + | − | − | − | − |
| ACC1024 | − | + | + | − | + | + | + |
| ACC1025 | − | + | + | − | − | − | − |
| ACC1026 | − | + | + | − | − | − | − |
| ACC1027 | − | + | + | − | + | + | + |
| ACC1028 | − | + | + | − | − | − | − |
| ACC1029 | − | + | + | − | + | + | + |
| ACC1030 | − | + | + | − | + | + | + |
| ACC1031 | − | + | + | − | + | + | + |
| ACC1032 | − | + | + | − | − | − | − |
| ACC1033 | − | + | + | − | + | + | + |
| ACC1034 | − | + | + | − | + | + | + |
| ACC1035 | − | + | + | − | + | + | + |
| ACC1036 | − | + | + | − | − | − | − |
| ACC1037 | − | + | + | − | + | + | + |
| ACC1038 | − | + | + | − | + | + | + |
| ACC1039 | − | + | + | − | + | + | + |
| ACC1040 | − | + | + | − | − | − | − |
| ACC1041 | − | + | + | − | − | − | − |
| ACC1042 | − | + | + | − | + | + | + |
| ACC1043 | − | + | + | − | − | − | − |
| ACC1044 | − | + | + | − | − | − | − |
| ACC1045 | − | + | + | − | + | + | + |
| ACC1046 | − | + | + | − | + | + | + |
| ACC1047 | − | + | + | − | + | + | + |
| ACC1048 | − | + | − | − | + | + | + |
| ACC1049 | − | + | − | − | − | − | − |
| ACC1050 | − | + | − | − | + | + | + |
| ACC1051 | − | + | − | − | − | − | − |
| ACC1052 | − | + | + | − | + | + | + |
| ACC1053 | − | + | + | + | + | + | + |
| ACC1054 | − | + | + | − | + | + | + |
| ACC1055 | − | + | + | − | + | + | + |
| ACC1056 | − | + | + | − | + | + | + |
| ACC1057 | − | + | + | − | + | + | + |
| ACC1058 | − | + | + | − | − | − | − |
| ACC1059 | − | + | + | − | − | − | − |
| ACC1060 | − | + | + | − | − | − | − |
| ACC1061 | − | + | − | − | + | + | + |
| ACC1062 | − | + | + | − | + | + | + |
| ACC1063 | − | + | + | − | − | − | − |
| ACC1064 | − | + | + | − | − | − | − |
| ACC1065 | − | + | + | − | − | − | − |
| ACC1066 | − | + | + | − | − | − | − |
| ACC1067 | − | + | + | − | − | − | − |
| ACC1068 | − | + | + | − | − | − | − |
| ACC1069 | − | + | − | − | + | + | + |
| ACC1070 | − | + | + | − | + | + | + |
| ACC1071 | − | + | + | − | − | − | − |
| ACC1072 | − | + | + | − | − | − | − |
| ACC1073 | − | + | + | − | − | − | − |
| ACC1074 | − | + | + | − | − | − | − |
| ACC1075 | − | + | + | − | − | − | − |
| ACC1076 | − | + | + | − | − | − | − |
| ACC1077 | − | + | − | − | + | + | + |
| ACC1078 | − | + | + | − | − | − | − |
| ACC1079 | − | + | + | − | + | + | + |
| ACC1080 | − | + | + | − | | | |
| ACC1081 | − | + | + | − | + | + | + |
| ACC1082 | − | + | + | − | − | − | − |
| ACC1083 | − | + | + | − | + | + | + |
| ACC1084 | − | + | + | − | + | + | + |
| ACC1085 | − | + | + | − | − | − | − |
| ACC1086 | − | + | + | − | + | + | + |
| ACC1087 | − | + | + | − | + | + | + |
| ACC1088 | − | + | + | − | + | + | + |
| ACC1089 | − | + | + | − | + | + | + |
| ACC1090 | − | + | + | − | + | + | + |
| ACC1091 | − | + | + | − | + | + | + |
| ACC1092 | − | + | + | − | + | + | + |
| ACC1093 | − | + | + | − | − | − | − |
| ACC1094 | − | + | + | − | + | + | + |
| ACC1095 | − | + | + | − | + | + | + |
| ACC1096 | − | + | + | − | + | + | + |
| ACC1097 | − | + | + | − | − | − | − |
| ACC1098 | − | + | + | − | + | + | + |
| ACC1099 | − | + | + | − | + | + | + |
| ACC1100 | − | + | + | − | + | + | + |
| ACC1101 | − | + | + | − | − | − | − |
| ACC1102 | − | + | + | − | + | + | + |
| ACC1103 | − | + | + | − | + | + | + |
| ACC1104 | − | + | + | − | + | + | + |
| ACC1105 | − | + | + | + | + | + | + |
| ACC1106 | − | + | + | + | − | − | − |
| ACC1107 | − | + | + | − | − | − | − |
| ACC1108 | − | + | + | − | + | + | + |
| ACC1109 | − | + | + | − | − | − | − |
| ACC1110 | − | + | + | − | − | − | − |
| ACC1111 | − | + | + | + | + | + | + |
| ACC1112 | − | + | + | + | − | − | − |

TABLE 4
Bacterial microbiomes from site MGSAMPBS001,
multiple traits and heavy metal tolerance

| | Plant growth promotion traits | | | | | | |
|---|---|---|---|---|---|---|---|
| | | ACC | | | Heavy Metal | | |
| MG Isolate | Abscisic Acid | deaminase | Nitrogen | Phosphate | tolerance (2 mM) | | |
| ID | Degradation | activity | fixation | solubilisation | Cd | Zn | Pb |
| CZL1113 | ND | ND | − | + | + | + | + |
| CZL1114 | ND | ND | − | − | + | + | + |
| CZL1115 | ND | ND | + | − | + | + | + |
| CZL1116 | ND | ND | + | − | + | + | + |
| CZL1117 | ND | ND | + | − | + | + | + |
| CZL1118 | ND | ND | + | − | + | + | + |
| CZL1119 | ND | ND | + | − | + | + | + |
| CZL1120 | ND | ND | − | − | + | + | + |
| CZL1121 | ND | ND | − | − | + | + | + |
| CZL1122 | ND | ND | + | − | + | + | + |
| CZL1123 | ND | ND | + | − | + | + | + |
| CZL1124 | ND | ND | + | − | + | + | + |
| CZL1125 | ND | ND | + | − | + | + | + |
| CZL1126 | ND | ND | + | − | + | + | + |
| CZL1127 | ND | ND | + | − | + | + | + |
| CZL1128 | ND | ND | − | − | + | + | + |
| CZL1129 | ND | ND | + | + | + | + | + |
| CZL1130 | ND | ND | + | − | + | + | + |
| CZL1131 | ND | ND | + | − | + | + | + |
| CZL1132 | ND | ND | − | − | + | + | + |
| CZL1133 | ND | ND | − | − | + | + | + |
| CZL1134 | ND | ND | + | − | + | + | + |
| CZL1135 | ND | ND | − | − | + | + | + |
| CZL1136 | ND | ND | + | − | + | + | + |
| CZL1137 | ND | ND | + | + | + | + | + |
| CZL1138 | ND | ND | + | − | + | + | + |
| CZL1139 | ND | ND | + | − | + | + | + |
| CZL1140 | ND | ND | + | − | + | + | + |
| CZL1141 | ND | ND | − | − | + | + | + |
| CZL1142 | ND | ND | − | − | + | + | + |
| CZL1143 | ND | ND | + | − | + | + | + |
| CZL1144 | ND | ND | + | − | + | + | + |
| CZL1145 | ND | ND | − | − | + | + | + |
| CZL1146 | ND | ND | − | − | + | + | + |
| CZL1147 | ND | ND | − | − | + | + | + |
| CZL1148 | ND | ND | + | − | + | + | + |
| CZL1149 | ND | ND | − | − | + | + | + |
| CZL1150 | ND | ND | − | − | + | + | + |
| CZL1151 | ND | ND | − | − | + | + | + |
| CZL1152 | ND | ND | + | − | + | + | + |
| CZL1153 | ND | ND | + | − | + | + | + |
| CZL1154 | ND | ND | − | − | + | + | + |
| CZL1155 | ND | ND | + | − | + | + | + |
| CZL1156 | ND | ND | + | − | + | + | + |
| CZL1157 | ND | ND | + | − | + | + | + |
| CZL1158 | ND | ND | + | − | + | + | + |
| CZL1159 | ND | ND | + | − | + | + | + |
| CZL1160 | ND | ND | + | − | + | + | + |
| CZL1161 | ND | ND | + | − | + | + | + |
| CZL1162 | ND | ND | − | − | + | + | + |
| CZL1163 | ND | ND | + | − | + | + | + |
| CZL1164 | ND | ND | + | − | + | + | + |
| CZL1165 | ND | ND | + | − | + | + | + |
| CZL1166 | ND | ND | + | − | + | + | + |
| CZL1167 | ND | ND | + | − | + | + | + |
| CZL1168 | ND | ND | + | − | + | + | + |
| CZL1169 | ND | ND | − | − | + | + | + |
| CZL1170 | ND | ND | + | − | + | + | + |
| CZL1171 | ND | ND | + | − | + | + | + |
| CZL1172 | ND | ND | + | − | + | + | + |
| CZL1173 | ND | ND | + | − | + | + | + |
| CZL1174 | ND | ND | + | − | + | + | + |
| CZL1175 | ND | ND | + | − | + | + | + |
| CZL1176 | ND | ND | − | − | + | + | + |
| CZL1177 | ND | ND | + | − | + | + | + |
| CZL1178 | ND | ND | + | − | + | + | + |
| CZL1179 | ND | ND | + | − | + | + | + |
| CZL1180 | ND | ND | + | − | + | + | + |
| CZL1181 | ND | ND | + | − | + | + | + |
| CZL1182 | ND | ND | + | − | + | + | + |
| CZL1183 | ND | ND | + | − | + | + | + |
| CZL1184 | ND | ND | − | − | + | + | + |
| CZL1185 | ND | ND | + | + | + | + | + |
| CZL1186 | ND | ND | + | − | + | + | + |
| CZL1187 | ND | ND | + | − | + | + | + |
| CZL1188 | ND | ND | + | − | + | + | + |
| CZL1189 | ND | ND | + | − | + | + | + |
| CZL1190 | ND | ND | + | − | + | + | + |
| CZL1191 | ND | ND | − | − | + | + | + |
| CZL1192 | ND | ND | + | − | + | + | + |
| CZL1193 | ND | ND | + | + | + | + | + |
| CZL1194 | ND | ND | + | − | + | + | + |
| CZL1195 | ND | ND | + | − | + | + | + |
| CZL1196 | ND | ND | + | − | + | + | + |
| CZL1197 | ND | ND | + | − | + | + | + |
| CZL1198 | ND | ND | − | − | + | + | + |
| CZL1199 | ND | ND | − | − | + | + | + |
| CZL1200 | ND | ND | + | − | + | + | + |
| CZL1201 | ND | ND | − | + | + | + | + |
| CZL1202 | ND | ND | − | − | + | + | + |
| CZL1203 | ND | ND | + | − | + | + | + |
| CZL1204 | ND | ND | + | − | + | + | + |
| CZL1205 | ND | ND | + | − | + | + | + |
| CZL1206 | ND | ND | + | − | + | + | + |
| CZL1207 | ND | ND | + | − | + | + | + |
| CZL1208 | ND | ND | + | − | + | + | + |

TABLE 5
Bacterial microbiomes from MGSAMPBS002,
multiple traits and heavy metal tolerance

| | Plant growth promotion traits | | | | | | |
|---|---|---|---|---|---|---|---|
| | | ACC | | | Heavy Metal | | |
| MG Isolate | Abscisic Acid | deaminase | Nitrogen | Phosphate | tolerance (2 mM) | | |
| ID | Degradation | activity | fixation | solubilisation | Cd | Zn | Pb |
| CZL1209 | ND | ND | − | + | + | + | + |
| CZL1210 | ND | ND | − | − | + | + | + |
| CZL1211 | ND | ND | − | − | + | + | + |
| CZL1212 | ND | ND | + | − | + | + | + |
| CZL1213 | ND | ND | − | − | + | + | + |
| CZL1214 | ND | ND | − | − | + | + | + |
| CZL1215 | ND | ND | − | − | + | + | + |
| CZL1216 | ND | ND | − | − | + | + | + |
| CZL1217 | ND | ND | − | − | + | + | + |
| CZL1218 | ND | ND | + | − | + | + | + |
| CZL1219 | ND | ND | + | − | + | + | + |
| CZL1220 | ND | ND | + | − | + | + | + |
| CZL1221 | ND | ND | + | − | + | + | + |
| CZL1222 | ND | ND | + | − | + | + | + |
| CZL1223 | ND | ND | − | − | + | + | + |
| CZL1224 | ND | ND | + | − | + | + | + |
| CZL1225 | ND | ND | + | − | + | + | + |
| CZL1226 | ND | ND | − | + | + | + | + |
| CZL1227 | ND | ND | + | + | + | + | + |
| CZL1228 | ND | ND | + | − | + | + | + |
| CZL1229 | ND | ND | + | − | + | + | + |
| CZL1230 | ND | ND | + | − | + | + | + |
| CZL1231 | ND | ND | − | + | + | + | + |
| CZL1232 | ND | ND | + | + | + | + | + |
| CZL1233 | ND | ND | − | + | + | + | + |
| CZL1234 | ND | ND | + | − | + | + | + |
| CZL1235 | ND | ND | + | + | + | + | + |
| CZL1236 | ND | ND | + | − | + | + | + |
| CZL1237 | ND | ND | + | − | + | + | + |
| CZL1238 | ND | ND | + | − | + | + | + |
| CZL1239 | ND | ND | − | − | + | + | + |
| CZL1240 | ND | ND | + | − | + | + | + |
| CZL1241 | ND | ND | − | − | + | + | + |
| CZL1242 | ND | ND | + | − | + | + | + |
| CZL1243 | ND | ND | + | − | + | + | + |
| CZL1244 | ND | ND | + | − | + | + | + |
| CZL1245 | ND | ND | + | − | + | + | + |
| CZL1246 | ND | ND | + | − | + | + | + |
| CZL1247 | ND | ND | + | − | + | + | + |
| CZL1248 | ND | ND | + | − | + | + | + |
| CZL1249 | ND | ND | + | − | + | + | + |
| CZL1250 | ND | ND | + | − | + | + | + |
| CZL1251 | ND | ND | + | − | + | + | + |
| CZL1252 | ND | ND | + | − | + | + | + |
| CZL1253 | ND | ND | + | − | + | + | + |
| CZL1254 | ND | ND | + | − | + | + | + |
| CZL1255 | ND | ND | + | − | + | + | + |
| CZL1256 | ND | ND | + | − | + | + | + |
| CZL1257 | ND | ND | + | − | + | + | + |
| CZL1258 | ND | ND | + | − | + | + | + |
| CZL1259 | ND | ND | + | − | + | + | + |
| CZL1260 | ND | ND | + | − | + | + | + |
| CZL1261 | ND | ND | + | − | + | + | + |
| CZL1262 | ND | ND | + | − | + | + | + |
| CZL1263 | ND | ND | − | − | + | + | + |
| CZL1264 | ND | ND | + | − | + | + | + |
| CZL1265 | ND | ND | + | − | + | + | + |
| CZL1266 | ND | ND | + | − | + | + | + |
| CZL1267 | ND | ND | + | − | + | + | + |
| CZL1268 | ND | ND | + | − | + | + | + |
| CZL1269 | ND | ND | + | − | + | + | + |
| CZL1270 | ND | ND | + | − | + | + | + |
| CZL1271 | ND | ND | + | + | + | + | + |
| CZL1272 | ND | ND | + | + | + | + | + |
| CZL1273 | ND | ND | + | − | + | + | + |
| CZL1274 | ND | ND | + | − | + | + | + |
| CZL1275 | ND | ND | + | − | + | + | + |
| CZL1276 | ND | ND | + | − | + | + | + |
| CZL1277 | ND | ND | + | − | + | + | + |
| CZL1278 | ND | ND | + | − | + | + | + |
| CZL1279 | ND | ND | + | − | + | + | + |
| CZL1280 | ND | ND | + | − | + | + | + |
| CZL1281 | ND | ND | + | − | + | + | + |
| CZL1282 | ND | ND | + | − | + | + | + |
| CZL1283 | ND | ND | + | − | + | + | + |
| CZL1284 | ND | ND | + | − | + | + | + |
| CZL1285 | ND | ND | + | − | + | + | + |
| CZL1286 | ND | ND | + | − | + | + | + |
| CZL1287 | ND | ND | + | − | + | + | + |
| CZL1288 | ND | ND | + | − | + | + | + |
| CZL1289 | ND | ND | + | − | + | + | + |
| CZL1290 | ND | ND | + | − | + | + | + |
| CZL1291 | ND | ND | + | − | + | + | + |
| CZL1292 | ND | ND | + | − | + | + | + |
| CZL1293 | ND | ND | + | − | + | + | + |
| CZL1294 | ND | ND | + | − | + | + | + |
| CZL1295 | ND | ND | + | − | + | + | + |
| CZL1296 | ND | ND | + | − | + | + | + |
| CZL1297 | ND | ND | + | − | + | + | + |
| CZL1298 | ND | ND | + | − | + | + | + |
| CZL1299 | ND | ND | + | − | + | + | + |
| CZL1300 | ND | ND | + | − | + | + | + |
| CZL1301 | ND | ND | + | − | + | + | + |
| CZL1302 | ND | ND | + | − | + | + | + |
| CZL1303 | ND | ND | + | − | + | + | + |
| CZL1304 | ND | ND | + | − | + | + | + |

ND: Trait was not determined

TABLE 6
Bacterial microbiomes isolated from MGSAMPBS010,
multiple traits and heavy metal tolerance

| | Plant growth promotion traits | | | | | | |
|---|---|---|---|---|---|---|---|
| | | ACC | | | Heavy Metal | | |
| MG Isolate | Abscisic Acid | deaminase | Nitrogen | Phosphate | tolerance (2 mM) | | |
| ID | Degradation | activity | fixation | solubilisation | Cd | Zn | Pb |
| CZL1305 | ND | ND | − | − | + | + | + |
| CZL1306 | ND | ND | | − | + | + | + |
| CZL1307 | ND | ND | + | − | + | + | + |
| CZL1308 | ND | ND | + | − | + | + | + |
| CZL1309 | ND | ND | + | − | + | + | + |
| CZL1310 | ND | ND | + | − | + | + | + |
| CZL1311 | ND | ND | + | − | + | + | + |
| CZL1312 | ND | ND | + | − | + | + | + |
| CZL1313 | ND | ND | + | − | + | + | + |
| CZL1314 | ND | ND | + | − | + | + | + |
| CZL1315 | ND | ND | + | − | + | + | + |
| CZL1316 | ND | ND | + | − | + | + | + |
| CZL1317 | ND | ND | + | − | + | + | + |
| CZL1318 | ND | ND | + | − | + | + | + |
| CZL1319 | ND | ND | + | − | + | + | + |
| CZL1320 | ND | ND | + | − | + | + | + |
| CZL1321 | ND | ND | | − | + | + | + |
| CZL1322 | ND | ND | + | − | + | + | + |
| CZL1323 | ND | ND | + | − | + | + | + |
| CZL1324 | ND | ND | + | − | + | + | + |
| CZL1325 | ND | ND | + | − | + | + | + |
| CZL1326 | ND | ND | + | − | + | + | + |
| CZL1327 | ND | ND | + | − | + | + | + |
| CZL1328 | ND | ND | + | − | + | + | + |
| CZL1329 | ND | ND | | − | + | + | + |
| CZL1330 | ND | ND | + | − | + | + | + |
| CZL1331 | ND | ND | + | − | + | + | + |
| CZL1332 | ND | ND | + | − | + | + | + |
| CZL1333 | ND | ND | + | − | + | + | + |
| CZL1334 | ND | ND | + | − | + | + | + |
| CZL1335 | ND | ND | + | − | + | + | + |
| CZL1336 | ND | ND | + | − | + | + | + |
| CZL1337 | ND | ND | | − | + | + | + |
| CZL1338 | ND | ND | | − | + | + | + |
| CZL1339 | ND | ND | | + | + | + | + |
| CZL1340 | ND | ND | + | − | + | + | + |
| CZL1341 | ND | ND | + | − | + | + | + |
| CZL1342 | ND | ND | + | − | + | + | + |
| CZL1343 | ND | ND | + | − | + | + | + |
| CZL1344 | ND | ND | + | − | + | + | + |
| CZL1345 | ND | ND | + | − | + | + | + |
| CZL1346 | ND | ND | + | − | + | + | + |
| CZL1347 | ND | ND | + | + | + | + | + |
| CZL1348 | ND | ND | + | − | + | + | + |
| CZL1349 | ND | ND | + | − | + | + | + |
| CZL1350 | ND | ND | + | − | + | + | + |
| CZL1351 | ND | ND | + | − | + | + | + |
| CZL1352 | ND | ND | + | − | + | + | + |
| CZL1353 | ND | ND | + | − | + | + | + |
| CZL1354 | ND | ND | + | − | + | + | + |
| CZL1355 | ND | ND | + | − | + | + | + |
| CZL1356 | ND | ND | + | − | + | + | + |
| CZL1357 | ND | ND | + | − | + | + | + |
| CZL1358 | ND | ND | + | − | + | + | + |
| CZL1359 | ND | ND | + | − | + | + | + |
| CZL1360 | ND | ND | + | − | + | + | + |
| CZL1361 | ND | ND | + | − | + | + | + |
| CZL1362 | ND | ND | + | − | + | + | + |
| CZL1363 | ND | ND | + | − | + | + | + |
| CZL1364 | ND | ND | + | − | + | + | + |
| CZL1365 | ND | ND | + | − | + | + | + |
| CZL1366 | ND | ND | + | − | + | + | + |
| CZL1367 | ND | ND | + | − | + | + | + |
| CZL1368 | ND | ND | + | − | + | + | + |
| CZL1369 | ND | ND | + | − | + | + | + |
| CZL1370 | ND | ND | + | − | + | + | + |
| CZL1371 | ND | ND | | − | + | + | + |
| CZL1372 | ND | ND | + | − | + | + | + |
| CZL1373 | ND | ND | + | − | + | + | + |
| CZL1374 | ND | ND | + | − | + | + | + |
| CZL1375 | ND | ND | + | − | + | + | + |
| CZL1376 | ND | ND | + | − | + | + | + |
| CZL1377 | ND | ND | + | − | + | + | + |
| CZL1378 | ND | ND | + | − | + | + | + |
| CZL1379 | ND | ND | + | − | + | + | + |
| CZL1380 | ND | ND | + | − | + | + | + |
| CZL1381 | ND | ND | + | − | + | + | + |
| CZL1382 | ND | ND | + | − | + | + | + |
| CZL1383 | ND | ND | + | − | + | + | + |
| CZL1384 | ND | ND | + | − | + | + | + |
| CZL1385 | ND | ND | − | − | + | + | + |
| CZL1386 | ND | ND | − | − | + | + | + |
| CZL1387 | ND | ND | − | − | + | + | + |
| CZL1388 | ND | ND | − | − | + | + | + |
| CZL1389 | ND | ND | + | − | + | + | + |
| CZL1390 | ND | ND | + | − | + | + | + |
| CZL1391 | ND | ND | + | − | + | + | + |
| CZL1392 | ND | ND | + | + | + | + | + |
| CZL1393 | ND | ND | + | − | + | + | + |
| CZL1394 | ND | ND | + | − | + | + | + |
| CZL1395 | ND | ND | + | − | + | + | + |
| CZL1396 | ND | ND | + | − | + | + | + |
| CZL1397 | ND | ND | + | − | + | + | + |
| CZL1398 | ND | ND | + | + | + | + | + |
| CZL1399 | ND | ND | + | − | + | + | + |
| CZL1400 | ND | ND | + | + | + | + | + |

Table 7 shows the percentage of the isolate with 1, 2, 3, 4, or 5 traits in each of the samples.

TABLE 7
Percentage of isolates from each site examined with multiple beneficial traits

| | | Percentage of isolates with multiple traits (%) | | | | |
|---|---|---|---|---|---|---|
| Sample Site | Source | 5 traits | 4 traits | 3 traits | 2 traits | 1 traits |
| MGSAMBSP001 | Soil | ND | ND | 4.17 | 68.75 | 27.08 |
| MGSAMPBS002 | Soil | ND | ND | 5.21 | 83.33 | 11.46 |
| MGSAMP005 | Plant | 11.07 | 36.16 | 38.75 | 11.07 | 2.95 |
| MGSAMP006 | Plant | 0.00 | 22.31 | 23.36 | 44.88 | 9.45 |
| MGSAMP008 | Plant | 20.22 | 6.96 | 43.04 | 25.22 | 4.57 |
| MGSAMBSP010 | Soil | ND | ND | 4.17 | 84.38 | 11.46 |

When isolating through the CFM process there is increased potential for the isolation of high numbers of microbes with multiple beneficial traits. This high throughput screening process captures and screens significantly higher numbers of isolates than traditional screening processes in a very short period of time. Results indicate that up to 20% of isolates from plant microbiomes had five multiple traits. Microbes isolated from the soil were examined for three traits. Through the CFM process up to 84% of isolates originating from the soil had two or more traits. With the potential to isolate up to 10,000 isolates, 2000 of these may have up to 5 beneficial traits, indicating that the process increases the probability of successfully isolating microbes that will function successfully in the environment.

A compressed library of 478 actively growing microbes that are tolerant to 2 mM CZL were isolated and stored for further testing. A library of 184 PO4 solubilising microbes were isolated and stored for further testing. From the microbiomes enriched for isolation of diazotrophs a library of 186 actively growing microbes were isolated. A total of 276 ACC degrading microbes were isolated.

Purification and Characterisation Isolates

A total of 478 microbes were isolated from the final enriched plant and soil functional microbiomes. 189 isolates were from plant microbiomes. These plant microbiome isolates were categorised by their ability to solubilise PO4 and their additional multiple traits. To determine the MIC values for each isolate, QTrays containing TG agar supplemented with increasing concentrations of Cd were inoculated using a 96 pin replicator and incubated for 48 hrs at 27° C. Bacterial growth was examined visually on Qtrays to determine Cd MIC.

There was 138 heavy metal tolerant isolates that were capable of solubilising PO4. 47.1% of these isolates had two or more multiple traits. There are 51 heavy metal isolates were not PO4 solubilisers. The non-PO4 solubilising isolates that showed higher Cd tolerance levels and had a number of multiple traits were purified by continually inoculating single colonies onto LB containing 2 mM Cd. Following purification isolates were tested for their multiple traits and 45 of these purified isolates were selected for 16s rRNA identification (Table 8).

TABLE 8
Purified heavy metal tolerant isolates. 16S identification. Cd MIC. urease activity and multiple traits.

| Isolate | | Sample | Cd MIC | | | | PO4 | | | | Urease | |
|---|---|---|---|---|---|---|---|---|---|---|---|---|
| label | Isolate ID | location | (mM) | Diazo | ACC | ABA | solubilisation | Gram | Oxidase | Catalase | 24 hrs | 48 hrs |
| MBPI001 | Sphingobacterium sp. | MGSAMP005 | 2 | + | + | + | − | − | + | − | ++ | ++++ |
| MBPI002 | Sphingobacterium sp. | MGSAMP005 | 2.5 | + | + | + | − | − | + | − | ++ | ++++ |
| MBPI003 | Sphingobacterium sp. | MGSAMP005 | 2.5 | + | + | + | − | − | + | − | ++ | ++++ |
| MBPI004 | Sphingobacterium sp. | MGSAMP005 | 2.5 | + | + | + | − | − | ND | ND | ++ | ++++ |
| MBPI005 | Sphingobacterium | MGSAMP005 | 2.5 | + | + | + | − | − | + | + | ++ | ++++ |
| | multivorum | | | | | | | | | | | |
| MBPI006 | Serratia sp. | MGSAMP005 | 3 | + | + | + | − | − | − | + | + | ++++ |
| MBPI007 | Serratia fonticola | MGSAMP005 | 2.5 | + | + | + | − | − | − | | ++ | ++++ |
| MBPI008 | Serratia fonticola | MGSAMP005 | 2 | + | + | + | − | − | + | + | + | ++++ |
| MBPI009 | Achromobacter | MGSAMP005 | 2 | + | + | + | − | − | + | + | ++ | +++ |
| | ruhlandii | | | | | | | | | | | |
| MBPI010 | Serratia fonticola | MGSAMP005 | 4 | + | + | + | − | − | + | + | + | +++ |
| MBPI011 | Serratia fonticola | MGSAMP005 | 2.5 | + | + | + | − | − | + | + | + | +++ |
| MBPI012 | Serratia sp. | MGSAMP005 | 2.5 | + | + | + | − | − | + | − | + | +++ |
| MBPI013 | Serratia fonticola | MGSAMP005 | 2.5 | + | + | + | − | − | + | − | ++ | ++++ |
| MBPI014 | Serratia sp. | MGSAMP005 | 2.5 | + | + | + | − | − | + | + | ++ | ++++ |
| MBPI015 | Serratia fonticola | MGSAMP005 | 2.5 | + | + | + | − | − | + | − | − | +++ |
| MBPI016 | Serratia fonticola | MGSAMP005 | 6 | + | + | + | − | − | − | + | − | +++ |
| MBPI017 | Serratia sp. | MGSAMP005 | 2 | + | − | + | − | − | − | + | − | +++ |
| MBPI018 | Stenotrophomonas | MGSAMP005 | 2.5 | + | − | + | − | − | − | + | + | +++ |
| | maltophilia | | | | | | | | | | | |
| MBPI019 | Serratia sp. | MGSAMP006 | 2.5 | + | + | + | − | − | − | + | − | +++ |
| MBPI020 | Serratia sp. | MGSAMP006 | 2.5 | + | + | + | − | | − | + | − | +++ |
| MBPI021 | Serratia fonticola | MGSAMP006 | 3 | + | + | + | | − | + | + | + | +++ |
| MBPI022 | Ochrobactrum | MGSAMP006 | 3 | + | + | + | ++ | − | + | + | ++ | +++ |
| | intermedium | | | | | | | | | | | |
| MBPI023 | Serratia fonticola | MGSAMP006 | 3 | + | + | − | − | − | − | + | − | +++ |
| MBPI024 | Serratia sp. | MGSAMP006 | 6 | + | + | − | − | − | − | + | − | +++ |
| MBPI025 | Serratia sp. | MGSAMP006 | 2 | + | − | + | + | − | − | + | − | +++ |
| MBPI026 | Serratia fonticola | MGSAMP006 | 2 | + | + | + | + | − | − | + | − | +++ |
| MBPI027 | Serratia sp. | MGSAMP006 | 3 | + | + | + | − | − | − | + | − | +++ |
| MBPI028 | Serratia fonticola | MGSAMP006 | 3 | + | + | + | − | − | − | + | − | +++ |
| MBPI029 | Serratia sp. | MGSAMP006 | 2.5 | + | + | + | − | − | − | + | − | +++ |
| MBPI030 | Serratia sp. | MGSAMP006 | 3 | + | + | − | − | | − | + | − | +++ |
| MBPI031 | Serratia sp. | MGSAMP006 | 3 | + | + | − | − | − | − | + | − | +++ |
| MBPI032 | Serratia fonticola | MGSAMP006 | 6 | + | + | + | − | − | − | + | − | +++ |
| MBPI033 | Serratia sp. | MGSAMP006 | 2.5 | + | + | + | + | − | − | + | + | ++++ |
| MBPI034 | Serratia sp. | MGSAMP006 | 2.5 | + | + | + | + | − | − | + | + | +++ |
| MBPI035 | Serratia sp. | MGSAMP006 | 2.5 | + | + | + | + | − | − | + | − | +++ |
| MBPI036 | Serratia sp. | MGSAMP006 | 2.5 | + | + | + | + | − | − | + | − | +++ |
| MBPI037 | Serratia fonticola | MGSAMP006 | 2.5 | + | + | + | − | − | − | + | + | +++ |
| MBPI038 | Chryseobacterium | MGSAMP006 | 3 | + | + | + | − | − | − | + | + | +++ |
| | bernardetii | | | | | | | | | | | |
| MBPI039 | Serratia sp. | MGSAMP006 | 2.5 | + | + | + | − | − | − | + | − | +++ |
| MBPI040 | Ochrobactrum sp. | MGSAMP008 | 4 | + | + | + | − | − | − | + | + | +++ |
| MBPI041 | Ochrobactrum | MGSAMP008 | 6 | + | + | + | + | − | + | + | + | +++ |
| | intermedium | | | | | | | | | | | |
| MBPI042 | Ochrobactrum | MGSAMP008 | 4 | + | + | + | − | − | − | + | + | +++ |
| | intermedium | | | | | | | | | | | |
| MBPI043 | Serratia fonticola | MGSAMP008 | 4 | + | + | + | − | − | − | + | − | +++ |
| MBPI044 | Ochrobactrum | MGSAMP008 | 4 | + | + | + | − | − | + | + | − | +++ |
| | intermedium | | | | | | | | | | | |
| MBPI045 | Chryseobacterium | MGSAMP008 | 6 | + | + | + | − | − | + | + | − | − |
| | bernardetii | | | | | | | | | | | |

Screening for Urease Induced Microbial Calcite Precipitation Ability

The capability of the selected isolates to produce urease was tested to screen potential isolate to stabilise cadmium. Bacteria that produces the enzyme urease can hydrolyze urea. Due to this enzymatic reaction, the pH of the media will increase and carbonate is produced resulting in mineralisation of the soluble heavy metal ions present in the media.

The 45 selected strains were cultured onto phenol red-urea agar plates. These plates were incubated at 30° C. and examined for growth and colour change after 24 and 48 hrs. A colour change from yellow to red/dark purple indicated carbonate production as a by product of urea hydrolysis.

As shown in Table 8, 24 out of the 45 isolates produce urease after 24 hrs, and 44 out of 45 produced the urease enzyme after 48 hrs incubation. These urease producing isolates had the potential capability to stabilise cadmium.

Determination of Cd Uptake by Bacterial Isolates

A laboratory-based bioassay was conducted to determine the levels of Cd uptake by the selected microbes. Bacterial isolates were inoculated into 100 ml LB broth containing 50 ppm cadmium and incubated at 28° C. for 24 hrs. To determine the levels of dissolved Cd, the bacterial cultures were centrifuged for 2 hrs at 2000 rpm, 10° C. The supernatant was filtered and acidified with nitric acid (final concentration 5.0%v/v) and analysed by AAS. Sterile broth with 50 ppm Cd was also acidified and analysed by AAS as a control. The lower the cadmium level in the supernatant, the higher the accumulation capability of the bacteria.

As shown in Table 9, the level of dissolved Cd in the supernatant was reduced for all isolates examined. bacterial isolate MBPI018 and MBPI024 showed greatest capability to stabiles cadmium which was seen 4.51 ppm and 3.41 ppm reduction in the supernatant, respectively.

TABLE 9
Level of Cd reduction uptake by bacterial isolates -
levels of dissolved Cd in supernatant

Supernatant

| MB # | Cd measured (ppm) | % reduction | ppm reduction |
|---|---|---|---|
| Control | 51.35 | — | — |
| PI004 | 50.14 | 2.36 | 1.21 |
| PI009 | 48.93 | 4.71 | 2.41 |
| PI010 | 49.56 | 3.49 | 1.79 |
| PI018 | 46.83 | 8.80 | 4.51 |
| PI024 | 47.93 | 6.66 | 3.41 |
| PI038 | 49.56 | 3.49 | 1.79 |
| PI041 | 49.40 | 3.72 | 1.94 |
| PI045 | 48.30 | 5.94 | 3.04 |

Bacterial Effects on Rice Plant Development

The effect of 45 bacterial isolates on the development of rice was investigated on rice germination and biomass of rice seeding.

The Effects of Bacterial Isolates on Rice Germination

Rice seeds were inoculated with bacteria by submerging in 24 hr cultures (bacterial concentration of 10⁸ CFU/ml) for 60 mins. Germination was assessed in 9 cm Petri dishes containing 20 ml of sterile dH₂O and 12 rice seeds. 1 ml of a 10⁸ CFU/ml bacterial culture was inoculated into each dish (final bacterial concentration 10⁶ CFU/ml). Replication was three fold per bacterial isolate. Seeds submerged and subsequently inoculated with LB broth were used as a control. Petri-dishes were sealed with parafilm to prevent excess evaporation. Seeds were incubated at 30° C. in the dark for 4 days after which germination rates were assessed.

The mean germination rate in the control was 63.89% (±SE 2.27). 8 out of the 45 isolates significantly increased the the germination rate by 10%. All isolates did not show significant negative effect on seed germination when compared to germination in the control samples.

Effects of Bacterial Isolates on Early Rice Seedling Development

Seven day old seedlings germinated in the presence of bacterial isolates (10⁶ CFU/ml) were examined to determine the effects of isolates on early seedling development. Following seven days incubation at 30° C. in the dark, seeds were photographed and the cumulative biomass of the seeds for each replicate was determined. The mean biomass per replicate was determined for each isolate and compared to the control to determine any negative or stimulatory affects.

9 out 45 of the bacterial isolate elicited up to 16% increase of seeding biomass after 7 days growth. The remaining 36 strains did not have any significant negative impact on the development of rice seedling.

Determination of Plant Growth Promotion and Cd Stabilisation Capability of the Isolates in Rice (Oryza sativa) Growing in Naturally Cadmium Contaminated Soil

To examine the effect of bacterial isolates on rice plant and Cd stabilization capability in soil naturally contaminated with Cd, five-day-old rice seedlings were planted in cadmium contaminated soil from Hunan province, China. 1 ml of a bacterial consortia consisting of eight isolates (Table 10) were inoculated into the pots with rice seedlings. 1 ml of sterile water was added to rice seedings in the control pots. Seedlings were grown in a growth chamber. 20 day old seedlings were harvested and the biomass was determined. Plants were dried and homogenised for digestion and the level of Cd in plant roots and leaves of inoculated plants and un-inoculated plants were measured by AAS.

TABLE 10
Bacterial consortia inoculated on rice seeding for Cd stabilisation

| | | Isolate site | Cd MIC | | | | PO4 | | | |
|---|---|---|---|---|---|---|---|---|---|---|
| Isolate No | Isolate ID | location | (mM) | Diazo | ACC | ABA | solubilisation | Gram | Oxidase | Catalase |
| MBPI001 | Sphingobacterium sp. | MGSAMP005 | 2 | + | + | + | − | − | + | − |
| MBPI002 | Sphingobacterium sp. | MGSAMP005 | 2.5 | + | + | + | − | − | + | − |
| MBPI005 | Sphingobacterium | MGSAMP005 | 2.5 | + | + | + | − | − | + | + |
| | multivorum | | | | | | | | | |
| MBPI010 | Serratia fonticola | MGSAMP005 | 4 | + | + | + | − | − | + | + |
| MBPI013 | Serratia fonticola | MGSAMP005 | 2.5 | + | + | + | − | + | + | − |
| MBPI014 | Serratia sp. | MGSAMP005 | 2.5 | + | + | + | − | − | + | + |
| MBPI016 | Serratia fonticola | MGSAMP005 | 6 | + | + | + | − | + | − | + |
| MBPI018 | Stenotrophomonas | MGSAMP005 | 2.5 | + | − | + | − | − | − | + |
| | maltophilia | | | | | | | | | |

The mean seedling length in bacterial treated plants was 128.6% greater than the length of the control plants. In addition, similar increases were recorded for the fresh weights of bacterial treated plants, here there was a 123.3% increase in fresh weights when compared to the control seedlings. AAS analysis showed promising results as Cd levels in root and leaf samples of un-inoculated plants (0.222 and 0.248, respectively) were higher than that of the inoculated root and leaf samples in the inoculated plants (0.006 and 0.006, respectively). Large scale greenhouse and field trials will be conducted to optimise microbial inoculants

The survival rate of the rice seedling in the bacteria inoculated pots was 83% and only 33% of the seedling survived in the non-inoculated pots. There was a 123.3% increase of fresh weight of the rice plant with the inoculation of bacterial consortium compared to without the inoculation. The mean seedling length in bacterial treated plants was 128.6% greater than the length of the control plants. AAS analysis showed Cd levels in root and leaf samples of un-inoculated plants are 0.222 and 0.248, respectively and Cd levels in the root and leaf samples of the bacteria inoculated rice seeding were 0.006 (±SE 0.003). 97% reduction of cadmium level in rice plant was observed in this experiment.

These results provide the evidence that the process referred as constructed functional microbiome, of the present invention is capable of producing microbes and microbial consortia with multiple plant specific beneficial trials for specific site need, in 3-6 months.

Furthermore, the microbes identified in this embodiment can be utilized to construct microbial products for promoting the growth of rice plants and reducing the accumulation of cadmium in rice pants to protect food safety.

Furthermore, the results also showed the microbial consortia existed in a cooperative or synergistic state which enhanced their ability to perform the beneficial traits, when applied to the same soil condition from where the microbial consortia were initially isolated. These microbial consortia can be developed as efficient commercial products for that particular site or sites with similar soil conditions.

### Example 2: Application of the Constructed Functional Microbiome Process to Identify Microorganisms Able to Promote Corn Yield in a Field Trial

Soil and plant samples were collected from a corn planted field in the North of China. Two endophytic bacterial strains with multiple beneficial traits were identified from corn plants by the application of the following constructed functional microbiome process:
Collecting corn plant samples, rhizosphere and bulk soil
Liberating any microorganisms present into a liquid medium
Constructing IAA producing functional microbiome by enriching the above microbial culture in DF growth media containing a mixture of IAA intermediates as the sole nitrogen source.
Constructing ACC producing functional microbiome by enriching the above IAA producing microbiome in DF growth media containing ACC hydrochloride as the sole nitrogen source.
Constructing ABA producing functional microbiome by enriching the ACC producing functional microbiome in DF growth media supplemented with 10 mg/l ABA as the sole carbon source. Constructing phosphate solubilising functional microbiome by enriching the ABA producing functional microbiome in National Botanical Research Institute's Phosphate (NBRIP) growth media containing tricalcium phosphate as the sole phosphate source.
Constructing diazotrophic functional microbiome by enriching the ABA producing functional microbiome in Combine Carbon Source (CCM) growth media containing 5 μg/ml biotin.
Isolating colonies of microorganisms from the above constructed functional microbiome with multiple beneficial traits
Without further isolation, testing the organisms from the colonies for one or more additional purifying the microbes with beneficial traits.
Testing the purified microbes with multiple beneficial traits

The best performing microbes, MB609 and MB806 with multiple traits, identified as Pseudomonas sp. and Azospirilium sp. by 16s RNA sequencing, were selected for demonstrating the promotion of corn yield in a field trial in north China.

The field trial was designed to compare the performance of microbial inoculated and non-inoculated corn seeds with the chemical fertiliser application rate of 100%, 70% and 50%.

Corn seeds were coated with mixture culture of MB609 (1×10⁷/ml) and MB806 (1×10⁷/ml). Coated and non-coated corn seeds were sown in plots of 10 by 7.5 meters arranged in a randomised block design. 8 replicate plots were sown for each treatment. After harvest, corn yield were measured for each treatment and shown in Table 11. At 100% application rate of chemical fertilisers, CMF selected microbes coated seed resulted in a 10.53% yield increase. With the reduction of 30% and 50% chemical application rate, corn seeds treated with the microbes resulted higher yield than the treatment with the non-inoculated seed at 100% fertiliser application rate.

TABLE 11
The yield of corn for each of the treatment

| | % of yield | % of yield | % of yield |
|---|---|---|---|
| | increased | increased | increased |
| | comparing non- | comparing non- | comparing non- |
| | coated seed + | coated seed + | coated seed + |
| Treatment | 100% fertiliser | 70% fertiliser | 50% fertiliser |
| coated seeds + | 10.53% | NR | NR |
| 100% fertiliser | | | |
| coated seeds + | 8.77% | 18.09% | NR |
| 70% fertiliser | | | |
| coated seeds + | 1.71% | NR | 16.32% |
| 50% fertiliser | | | |

NR: not relevant

This result provides evidence that the process of the present invention is capable of identifying microbes to promote plant growth in a field, and to reduce the chemical fertiliser application for beneficial economical and environmental impact.

### Example 3: Application of Site-Specific Functional Microbiome to Degrade Toxic Organic Compounds and Promote the Growth of Ryegrass Under Saline Soil Conditions

Wild plants samples and bulk soil were collected from 5 crude oil impacted agricultural fields in China. The average level of total petroleum hydrocarbon (TPH) in the soil was 20,000 ppm analysed by an independent accredited laboratory. The average salt level was 1.5%. The collected soil and plant samples were used as the source of constructing the functional microbiome and identifying microbes with multiple traits. The process was shown below,
Collecting corn plant samples, rhizosphere and bulk soil
Liberating any microorganisms present in the wild plants, rhizosphere and bulk soil into a liquid medium
Constructing TPH degrading functional microbiomes by enriching the above culture in Dworkin and Foster minimal broth media supplemented crude oil extract. The enriched culture was subsequently enriched in the same media for another 2-5 rounds.
Constructing IAA producing functional microbiome by enriching the above TPH degrading microbial culture in DF growth media containing a mixture of IAA intermediates as the sole nitrogen source.
Constructing ACC producing functional microbiome by enriching the above IAA producing microbiome in DF growth media containing ACC hydrochloride as the sole nitrogen source.
Constructing ABA producing functional microbiome by enriching the ACC producing functional microbiome in DF growth media supplemented with 10 mg/l ABA as the sole carbon source. Constructing phosphate solubilation functional microbiome by enriching the ABA producing functional microbiome in National Botanical Research Institute's Phosphate (NBRIP) growth media containing tricalcium phosphate as the sole phosphate source.
Constructing diazotrophic functional microbiome by enriching the ABA producing functional microbiome in Combine Carbon Source (CCM) growth media containing 5 μg/ml biotin.
Constructing salt resistant functional microbiome by enriching diazotrophic functional microbiome into nutrient broth supplemented with the 150 mM NaCl.
Constructing a final TPH degrading functional microbiome by enriching the above culture in Dworkin and Foster minimal broth media supplemented crude oil extract. (apply to soil pot planting with rye grass)
Isolating colonies of microorganisms from the above constructed functional microbiome with multiple beneficial traits.
Without further isolation, testing the organisms from the colonies for one or more additional purifying the microbes with beneficial traits.
Testing the purified microbes with multiple beneficial traits

42 bacterial strains with multiple traits were identified by 16s rRNA sequencing as shown in Table 12

TABLE 12
Identification of the isolated microbes

| MB# | ID |
|---|---|
| MB3C10 | Bacillus subterraneus |
| MB3E10 | Bacillus nealsonii |
| MB3F10 | Sphingomonas sp. |
| MB3H10 | Alcaligenes sp. |
| MB3A11 | Sphingobium sp. |
| MB3D11 | Bacillusn thuringiensis |
| MB3G11 | Pseudomonas stutzeri |
| MB3E12 | Bacillus thuringiensis |
| MB4A01 | Pseudomonas corrugata |
| MB4B01 | Pseudomonas thivervalensis |
| MB4H01 | Pseudomonas stutzeri |
| MB4B02 | Bacillus sp. |
| MB4C02 | Bacillus cereus |
| MB4H02 | Pseudomonas fluorescens |
| MB4D03 | Sphingobium sp. |
| MB4F04 | Bacillus pumilus |
| MB4G04 | Pseudomonas stutzeri |
| MB4A05 | Bacillus sp. |
| MB4B05 | Pseudomonas veronii |
| MB4G06 | Pseudomonas xanthomarina |
| MB4H06 | Pseudomonas synxantha |
| MB4A07 | Pseudomonas brassicacearum |
| MB4B07 | Pseudomonas thivervalensis |
| MB4C07 | Pseudomonas veronii |
| MB4D07 | Pseudomonas veronii |
| MB4E07 | Pseudomonas fluorescens |
| MB4F07 | Pseudomonas corrugata |
| MB4G07 | Pseudomonas brassicacearum |
| MB4H07 | Pseudomonas stutzeri |
| MB4A08 | Pseudomonas sp. |
| MB4C08 | Pseudomonas extremaustralis |
| MB4B09 | Pseudomonas marginalis |
| MB4E09 | Pseudomonas veronii |
| MB4F09 | Pseudomonas veronii |
| MB4G10 | Pseudomonas veronii |
| MB4E11 | Pseudomonas xanthomarina |
| MB4F11 | Pseudomonas sp. |
| MB4B12 | Pseudomonas |
| MG4D12 | Pseudomonas marginalis |
| MB4E12 | Pseudomonas veronii |
| MB4F12 | Pseudomonas marginalis |

ID: putative ID based on the closest match in RDPII database to 16s rRNA sequence

A greenhouse trial was set up testing the capability of the the microbes and the functional microbiome to degrade TPH and promote the growth of perennial ryegrass (Lolium perenne L.). 20 kg of TPH-contaminated soil from the same sites in China was air dried for 24 hrs and passed through a 2 mm sieve, to remove pebbles and debris. The sieved soil was homogenised by mixing. 20 pots containing lkg TPH contaminated soil were set up. The final enriched functional microbiome and two microbial consortium were used for inoculation. Microbial consortia 1 contained MB3C10, MBF3F10, MB3H10, MB3H02, MB4E09, MB4G07, isolated from the same TPH contaminated soil. Microbial consortia 2 contained 6 microbes with TPH degradation and plant growth promotion traits, MB0113, MB0321, MBA004, MBS001, MBS007, MBS129, were selected from MicroGen Biotech microbial collection library. 5 pots were inoculated the final enriched TPH degrading functional microbiome culture at an application rate of 10⁷/g soil and 15 ryegrass seeds coated with the same functional microbiome culture were sown in each of the pots; 5 pots were inoculated with microbial consortium 1, isolated from the contaminated soil at the application rate of 10⁷/g soil and 15 ryegrass seeds coated with the same microbial consortia were sown in each of the pots; 5 pots were inoculated with microbial consortia 2 and 15 ryegrass seeds coated with the same microbial consortia were sown in each of the pots; The remaining 5 pots were used as control without microbial inoculation and with 15 ryegrass sown into each pots. The plants were cultivated under greenhouse conditions (16 hrs at 24° C., 8 hr at 16° C.) for 12 weeks. Each pot was watered with three times per week. At the end of the experiment plant biomass, the concentration of the TPH in the plants and soil were measured in each of the pots. The results were shown in Table 13. The growth of ryegrass plants in the control pots were significantly stressed due to high salt and and high TPH contamination level in the soil. There was no accumulation of TPH in the ryegrass plant above ground level. The microbes introduced to the soil degraded higher level of TPH comparing to the control. The growth of the ryegrass plants was significantly increased when inoculated with the constructed functional microbiome and the microbial consortia as shown in Table 13, with the sequential of functional microbiome>consortia 1>consortia 2.

TABLE 13
The impact of microbial inoculation for ryegrass
growth and TPH degradation in soil

| | | | TPH level |
|---|---|---|---|
| | Biomass increased | Soil THP | in the plants |
| Inoculation | over control | reduction | above ground |
| TPH functional | 92.23% | 46.12% | 0 |
| microbiome | | | |
| microbial consortia 1 | 67.95% | 35.42% | 0 |
| Microbial consortia 2 | 25.75% | 14.84% | |
| No inoculation | N/A | 3.21% | N/A |

This result provides evidence that the process of the invention is capable of identifying microbes to promote ryegrass growth and degrade TPH in contaminated soil.

Furthermore, the results showed the microbial consortia and the functional microbiome showed enhanced ability to perform the beneficial traits, when applied in the same soil conditions from where the microbes were initially enriched. The microbial consortia can be developed as efficient commercial products for that particular site or sites with similar soil condition.

1. A method of constructing a functional microbiome comprising microbes with one or more beneficial traits, the method comprising:
(a) collecting one or more plant, rhizosphere or bulk soil samples from one or more agricultural or potential agricultural sites; the plant samples comprising at least one of the root, rhizome, shoot, flower, seed, seedling, fruit, stem, cuttings or leaves, or the soil attached to the plant,
(b) liberating any microorganisms present into a liquid medium,
(c) culturing any microorganisms present into an enrichment liquid medium to enrich functional microbiomes with one or more specific beneficial traits,
(d) plating out the functional microbiome with a desirable trait on a solid selection medium and selecting isolates for testing.

2. A method as claimed in claim 1 wherein the functional microbiome identified in step (c) may go through a series of sequential or parallel enrichment steps, with each enrichment step selecting for the same or a further additional trait, so that the constructed functional microbiome has one or multiple desirable traits.

3. A method as claimed in claim 1 or claim 2 wherein the isolate is purified, and one or more microbes with one or more specific beneficial traits are selected, or wherein the isolate is not purified and the isolates with desired traits are selected.

4. A method as claimed in claim 1 wherein the construction of the functional microbiome is site specific.

5. A method as claimed in claim 1 wherein at least two of the root, rhizome, shoot, flower, seed, fruit, stem and leaves of the plant are sampled.

6. A method as claimed in claim 1 wherein the functional microbiome 1 s constructed with a first most desirable trait.

7. A method as claimed in claim 1 wherein the method comprises one or more subsequent sequential enrichments, with each enrichment step selecting for a further additional trait, the resultant functional microbiome having multiple traits.

8. A method as claimed in claim 1 wherein the plant, rhizosphere and/or soil samples are collected from the area in which the constructed functional microbiome or the isolated microbes are ultimately to be used.

9. A method as claimed in claim 1 wherein the beneficial traits are selected from the group comprising inorganic and organic phosphate and potassium release; Diazotrophic (nitrogen fixing) activity; Plant hormone production (indole-3-acetic acid, cytokinins, giberillins); Plant stress hormone reduction and the reduction in the level of abscisic acid in the plant roots, the ability to degrade toxic organic compounds, the ability to sequester, accumulate, solubilise or immobilise toxic heavy metals and the ability to survive and grow in high saline and draught conditions.

10. A method as claimed in claim 1 in which a functional microbiome with one or more specific traits is selected, or alternatively one or more non-purified microorganisms with one or more multiple traits are selected, or alternatively one or more purified microbes with one or more multiple traits are selected.

11. A method as claimed any claim 1 wherein microbes within the extracted microbiome that possess specific traits are selectively enriched in liquid cultures to construct functional microbiomes, preferably wherein an enrichment step is carried out which enriches microbes selected from the group comprising phosphate solubilising microbes, IAA producing microbes, ACC deaminase producing microbes, diazotrophic microbes, abscisic acid degrading microbes, organic pollutant degrading microbes, heavy metal resistance microbes, salt resistance microbes.

12. A method as claimed in claim 1 wherein the beneficial traits are selected from:
(a) plant growth promotion traits selected from the group comprising ACC deaminase activity, inorganic phosphate solubilisation, organic phosphate liberation, indole-3-acetic acid production, abscisic acid degradation, diazotrophic activity, exopolysaccharide production;
(b) xenobiotic degradation traits selected from the group comprising crude oil, polycyclic aromatic hydrocarbons, phosphonate herbicides, triazine herbicides, nitroaromatics, chlorinated aromatic, volatile organic compounds, PCBs, dioxin/furans or cyanide;
(c) biocontrol traits selected from phenyacetic acid, 2,4 diacetylphloroglucinol and phenazine; and/or
(d) heavy metal tolerance, solubilisation or immobilisation traits selected from the group compnsmg cadmium, lead, chromium, nickel, copper, zinc, cobalt, mercury and arsenic tolerance.

13. A functional microbiome with one or more traits identified by a method as claimed in claim 1, or one or more purified microorganisms identified or isolated by a method as claimed in any preceding claim.

14. A composition comprising a functional microbiome or one or more microorganisms according to claim 13.

15. A method to impart beneficial traits to a plant or groups of plants, or to soil or to bioremediation comprising administering to the plant, or groups of plants, or to the soil the composition of claim 14.
